(19) United States
(12) Patent Application Publication
(10) Pub. No.: US 2011/0229469 A1
(43) Pub. Date: Sep. 22, 2011

(54) METHODS FOR THE TREATMENT OF CANCER

(75) Inventor: Terrance Johns, Victoria (AU)

(73) Assignee: LUDWIG INSTITUTE FOR CANCER RESEARCH, New York, NY (US)

(21) Appl. No.: 13/122,106

(22) PCT Filed: Sep. 30, 2009
(86) PCT No.: PCT/US2009/005421

§ 371 Date: Jun. 13, 2011

Related U.S. Application Data
(60) Provisional application No. 61/101,971, filed on Oct. 1, 2008.

(51) Int. Cl. A61K 39/395; A61P 35/00; A61P 35/02

(52) U.S. Cl. 424/133.1; 424/138.1

(57) ABSTRACT

Methods for treating cancer with at least one HGF-Met inhibitor and at least one EGFR inhibitor are provided

This application claims the benefit of U.S. Provisional Application No. 61/101,971, filed Oct. 1, 2008, which is incorporated by reference herein.

### FIELD

The present invention relates to methods of treating cancer with an HGF-Met inhibitor and an EGFR inhibitor. Compositions and methods of producing said compositions are also described.

### BACKGROUND

Hepatocyte Growth Factor (HGF; also referred to in the literature as Scatter Factor (SF)) is a multifunctional heterodimeric polypeptide produced primarily by mesenchymal cells. HGF acts as a ligand for the Met receptor tyrosine kinase (Met). The human Met receptor is also known as “c-met.” Activation of the HGF-Met pathway has been shown to lead to an array of cellular responses, including, but not limited to proliferation (mitosis), scattering (motility), stimulation of cell movement through a matrix (invasion), and branching morphogenesis. The HGF-Met pathway plays a role in, e.g., neural induction, liver regeneration, wound healing, angiogenesis, growth, invasion, morphologic differentiation, and normal embryological development.

The epidermal growth factor receptor (EGFR) is a receptor tyrosine kinase receptor that is bound by a number of ligands. Activation of the EGFR pathway has been shown to lead to numerous cellular responses, including proliferation. The EGFRvIII protein is a mutant EGFR protein that contains a truncated extracellular have reported that although the EGFRvIII protein does not appear to bind any known ligands, it displays a low level of constitutive activation. See, e.g., Kuan et al., Endocrine-Related Cancer 8: 83-96 (2001).

Both aberrant HGF-Met pathway activity and aberrant EGFR pathway activity have been shown to be involved in tumorigenesis. EGFRvIII has been reported to be expressed in several types of tumors, including glioblastomas. See, e.g., Kuan et al., Endocrine-Related Cancer 8: 83-96 (2001).

The involvement of the HGF-Met and EGFR pathways in tumorigenesis suggested that methods of inhibiting those pathways might be useful in treating cancer.

### SUMMARY

In certain embodiments, methods for treating a resistant cancer in a patient comprising administering at least one HGF-Met inhibitor and at least one EGFR inhibitor are provided. In certain embodiments, the cancer expresses EGFRvIII.

In certain embodiments methods for treating a resistant cancer in a patient comprising administering: (i) at least one HGF-Met inhibitor and at least one EGFR inhibitor; and (ii) at least one chemotherapy treatment, are provided.

In certain embodiments methods for treating a resistant cancer in a patient comprising administering: (i) at least one HGF-Met inhibitor and at least one EGFR inhibitor; and (ii) at least one radiation treatment, are provided.

In certain embodiments, kits comprising at least one HGF-Met inhibitor and at least one EGFR inhibitor are provided.

Other embodiments of this invention will be readily apparent from the disclosure provided herewith.

### BRIEF DESCRIPTION OF THE FIGURES

FIG. 1A shows a plot of percent survival vs. days for mice inoculated with U87MG tumor cells and injected with 2.12.1 according to the work discussed in Example 1.

FIG. 1B shows a plot of tumor volume vs. days post inoculation for mice inoculated with U87MG tumor cells and injected with 2.12.1 according to the work discussed in Example 1.

FIG. 2A shows a plot of percent survival vs. days for mice inoculated with U87MGΔ2-7 tumor cells and injected with 2.12.1, panitumumab, or both 2.12.1 and panitumumab according to the work discussed in Example 2.

FIG. 2A shows a plot of tumor volume vs. days post inoculation for mice inoculated with U87MGΔ2-7 tumor cells and injected with 2.12.1, panitumumab, or both 2.12.1 and panitumumab according to the work discussed in Example 2.

FIG. 3 shows a plot of tumor volume vs. days post inoculation for mice inoculated with U87MGΔ2-7 tumor cells and injected with 2.12.1, panitumumab, or both 2.12.1 and panitumumab according to the work discussed in Example 3.

FIG. 4 shows a plot of tumor volume vs. days post inoculation for mice inoculated with U87MG.wt tumor cells and injected with 2.12.1 according to the work discussed in Example 4.

### DETAILED DESCRIPTION OF CERTAIN EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also the use of the term “portion” may include part of a moiety or the entire moiety.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

### Certain Definitions

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “hepatocyte growth factor” or “HGF” refers to a polypeptide as set forth in Nakamura et al., Nature 342: 440-443 (1989) or fragments thereof, as well as related polypeptides, which include, but are not limited to, allelic variants, splice variants, derivative variants, substitution variants, deletion variants, and/or insertion variants, fusion polypeptides, and interspecies homologs. In certain embodiments, an HGF polypeptide includes terminal residues, such as, but not limited to, leader sequence residues, targeting residues, amino terminal methionine residues, lysine residues, tag residues and/or fusion protein residues.

The term “Met” refers to a protein encoded by the nucleotide sequence set forth in Park et al., Proc. Natl. Acad. Sci. 84, 7479—(1987), or fragments thereof, as well as related polypeptides, which include, but are not limited to, allelic variants, splice variants, derivative variants, substitution variants, deletion variants, and/or insertion variants, fusion polypeptides, and interspecies homologs. In certain embodiments, a Met polypeptide includes terminal residues, such as, but not limited to, leader sequence residues, targeting residues, amino terminal methionine residues, lysine residues, tag residues and/or fusion protein residues.

The term “epidermal growth factor receptor” or “EGFR” refers to a polypeptide as set forth in Ullrich et al., Nature 6: 418-415 (1984) or fragments thereof, as well as related polypeptides, which include, but are not limited to, allelic variants, splice variants, derivative variants, substitution variants, deletion variants, and/or insertion variants, fusion polypeptides, and interspecies homologs. In certain embodiments, an EGFR polypeptide includes terminal residues, such as, but not limited to, leader sequence residues, targeting residues, amino terminal methionine residues, lysine residues, tag residues and/or fusion protein residues.

The term “EGFRvIII” refers to a polypeptide as set forth in Wikstrand et al., Journal of Neurovirology 4: 148-158 (1998).

The term “HGF-Met activity” includes any biological activity resulting from activation of the HGF-Met pathway. Exemplary activities include, but are not limited to, neural induction, liver regeneration, wound healing, growth, invasion, morphologic differentiation, embryological development, scattering, proliferation, apoptosis, cell motility, metastasis, migration, cell adhesion, integrin clustering, phosphorylation of paxillin, formation of focal adhesions, and cancer resulting from aberrant Met-HGF signaling. In certain embodiments, HGF-Met activity results from binding of HGF to Met.

The term “aberrant HGF-Met activity” includes any circumstance in which HGF-Met activity is either higher or lower than it should be. In certain circumstances, aberrant HGF-Met activity results from a concentration of HGF that is higher than it should be. In certain embodiments, aberrant HGF-Met activity results from a concentration of HGF that is lower than it should be. In certain circumstances, aberrant HGF-Met activity results from a concentration of Met that is higher than it should be. In certain embodiments, aberrant HGF-Met activity results from a concentration of Met that is lower than it should be. Aberrant Met-HGF activity can result, for example, in certain cancers.

The term “EGFR activity” includes any activity resulting from activation of the EGFR pathway. Exemplary activities include, but are not limited to, cell proliferation. In certain circumstances, EGFR activity results from binding of an EGFR ligand to EGFR. In certain circumstances, EGFR activity results from EGFRvIII.

The term “aberrant EGFR activity” includes any circumstance in which EGFR activity is either higher or lower than it should be. In certain embodiments, aberrant EGFR activity results from a concentration of EGFR that is higher than it should be. In certain embodiments, aberrant EGFR activity results from a concentration of EGFR that is lower than it should be. In certain circumstances, aberrant EGFR activity results from EGFRvIII. Aberrant EGFR activity can result, for example, in certain cancers.

The term “specific binding agent” refers to a natural or non-natural molecule that specifically binds to a target. Examples of specific binding agents include, but are not limited to, proteins, peptides, nucleic acids, carbohydrates, lipids, and small molecule compounds. In certain embodiments, a specific binding agent to HGF is an immunoglobulin. In certain embodiments, a specific binding agent to HGF is an immunoglobulin fragment. In certain embodiments, a specific binding agent is an antibody. In certain embodiments, a specific binding agent is an antigen binding region.

The term “specifically binds” refers to the ability of a specific binding agent to bind to a target with greater affinity than it binds to a non-target. In certain embodiments, specific binding refers to binding for a target with an affinity that is at least 10, 50, 100, 250, 500, or 1000 times greater than the affinity for a non-target. In certain embodiments, affinity is determined by an affinity ELISA assay. In certain embodiments, affinity is determined by a BIAcore assay. In certain embodiments, affinity is determined by a kinetic method. In certain embodiments, affinity is determined by an equilibrium/solution method.

The term “specific binding agent to HGF” refers to a specific binding agent that specifically binds any portion of HGF. In certain embodiments, a specific binding agent to HGF is an antibody. In certain embodiments, a specific binding agent to HGF is an antigen binding region.

The term “specific binding agent to Met” refers to a specific binding agent that specifically binds any portion of Met. In certain embodiments, a specific binding agent to Met is an antibody. In certain embodiments, a specific binding agent is an antigen binding region.

The term “specific binding agent to EGFR” refers to a specific binding agent that specifically binds any portion of EGFR or EGFRvIII. In certain embodiments, a specific binding agent to EGFR is an antibody. In certain embodiments, a specific binding agent to EGFR is an antigen binding region.

The term “HGF-Met inhibitor” refers to any molecule that leads to decreased HGF-Met activity. In certain embodiments an HGF-Met inhibitor is an HGF inhibitor. In certain embodiments an HGF-Met inhibitor is a Met inhibitor. In certain embodiments, an HGF-Met inhibitor is a specific binding agent. In certain embodiments, an HGF-Met inhibitor is an antibody.

The term “EGFR inhibitor” refers to any molecule that leads to decreased EGFR activity. In certain embodiments, an EGFR inhibitor is a specific binding agent. In certain embodiments, an EGFR inhibitor is an antibody.

The term “resistant cancer” refers to a cancer in which administration of 2.12.1 results in a greater tumor volume than the administration of an HGF-Met inhibitor and an EGFR inhibitor. In certain embodiments, a resistant cancer displays aberrant EGFR activity. In certain embodiments, a resistant cancer expresses EGFRvIII. In certain embodiments, a resistant cancer is a glioblastoma.

The terms “antibody” and “antibody peptide(s)” refer to an intact antibody, or a fragment thereof. In certain embodiments, the fragment includes contiguous portions of an intact antibody. In certain embodiments, the fragment includes non-contiguous portions of an intact antibody. In certain embodiments, the antibody fragment may be a binding fragment that competes with the intact antibody for specific binding. The term “antibody” also encompasses polyclonal antibodies and monoclonal antibodies. In certain embodiments, binding fragments are produced by recombinant DNA techniques. In certain embodiments, binding fragments are produced by enzymatic or chemical cleavage of intact antibodies. Binding fragments include, but are not limited to, Fab, Fab′, F(ab′)2, Fv, scFv, maxibodies, and single-chain antibodies. Non-antigen binding fragments include, but are not limited to, Fc fragments. The term “antibody” also encompasses anti-idiotypic antibodies that specifically bind to the variable region of another antibody. In certain embodiments, an anti-idiotypic antibody specifically binds to the variable region of an anti-HGF antibody. In certain embodiments, anti-idiotypic antibodies may be used to detect the presence of a particular anti-HGF antibody in a sample or to block the activity of an anti-HGF antibody.

The term “polyclonal antibody” refers to a heterogeneous mixture of antibodies that bind to different epitopes of the same antigen.

The term “monoclonal antibodies” refers to a collection of antibodies encoded by the same nucleic acid molecule. In certain embodiments, monoclonal antibodies are produced by a single hybridoma or other cell line, or by a transgenic mammal. Monoclonal antibodies typically recognize the same epitope. The term “monoclonal” is not limited to any particular method for making an antibody.

“Chimeric antibody” refers to an antibody that has an antibody variable region of a first species fused to another molecule, for example, an antibody constant region of another second species. See, e.g., U.S. Pat. No. 4,816,567 and Morrison et al., Proc Natl Aced Sci (USA), 81:6851-6855 (1985). In certain embodiments, the first species may be different from the second species. In certain embodiments, the first species may be the same as the second species. In certain embodiments, a chimeric antibody is a CDR-grafted antibody.

The term “CDR-grafted antibody” refers to an antibody in which the CDR from one antibody is inserted into the framework of another antibody. In certain embodiments, the antibody from which the CDR is derived and the antibody from which the framework is derived are of different species. In certain embodiments, the antibody from which the CDR is derived and the antibody from which the framework is derived are of different isotypes.

The term “multi-specific antibody” refers to an antibody wherein two or more variable regions bind to different epitopes. The epitopes may be on the same or different targets. In certain embodiments, a multi-specific antibody is a “bi-specific antibody,” which recognizes two different epitopes on the same or different antigens.

The term “catalytic antibody” refers to an antibody in which one or more catalytic moieties is attached. In certain embodiments, a catalytic antibody is a cytotoxic antibody, which comprise a cytotoxic moiety.

The term “humanized antibody” refers to an antibody in which all or part of an antibody framework region is derived from a human, but all or part of one or more CDR regions is derived from another species, for example a mouse.

The term “fully human antibody” refers to an antibody in which both the CDR and the framework comprise substantially human sequences. In certain embodiments, fully human antibodies are produced in non-human mammals, including, but not limited to, mice, rats, and lagomorphs. In certain embodiments, fully human antibodies are produced in hybridoma cells. In certain embodiments, fully human antibodies are produced recombinantly.

The term “anti-idiotype antibody” refers to an antibody that specifically binds to another antibody.

The term “heavy chain” includes any polypeptide having sufficient variable region sequence to confer specificity for a target. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carboxy-terminus. The term “heavy chain”, as used herein, encompasses a full-length heavy chain and fragments thereof.

The term “light chain” includes any polypeptide having sufficient variable region sequence to confer specificity for a target. A full-length light chain includes a variable region domain, V_{L}, and a constant region domain, C_{L}. Like the heavy chain, the variable region domain of the light chain is at the amino-terminus of the polypeptide. The term “light chain”, as used herein, encompasses a full-length light chain and fragments thereof.

The term “Fab fragment” refers to an antibody comprising one light chain and the C_{H}1 and variable regions of one heavy chain. The heavy chain of a Fab fragment cannot form a disulfide bond with another heavy chain. In certain embodiments, the heavy chain of a Fab fragment forms a disulfide bond with the light chain of a Fab fragment.

The term “Fab′ fragment” refers to an antibody comprising one light chain, the variable and C_{H}1 regions of one heavy chain, and some of the constant region between the C_{H}1 and C_{H}2 domains of the heavy chain. In certain embodiments, an interchain disulfide bond can be formed between two heavy chains of an Fab′ fragment to form a F(ab′)₂ molecule.

The term “F(ab′)₂ molecule” refers to an antibody comprising two Fab′ fragments connected by an interchain disulfide bond formed between two heavy chains.

An “Fv molecule” comprises the variable regions from both the heavy and light chains, but lacks the constant regions. A single chain variable fragment (scFv) comprises variable regions from both a heavy and a light chain wherein the heavy and light chain variable regions are fused to form a single polypeptide chain which forms an antigen-binding region. In certain embodiments, a scFV comprises a single polypeptide chain. A single-chain antibody comprises a scFV. In certain embodiments, a single-chain antibody comprises one or more additional polypeptides fused to a scFv. Exemplary additional polypeptides include, but are not limited to, one or more constant regions. Exemplary single-chain antibodies are discussed, e.g., in WO 88/01649 and U.S. Pat. Nos. 4,946,778 and 5,260,203.

The term “maxibody” refers to a scFv fused (may be by a linker or direct attachment) to an Fc or an Fc fragment. In certain embodiments, a single chain antibody is a maxibody. In certain embodiments, a single chain antibody is a maxibody that binds to HGF. Exemplary Ig-like domain-Fc fusions are disclosed in U.S. Pat. No. 6,117,655.

An “Fc fragment” comprises the C_{H}2 and C_{H}3 domains of the heavy chain and contains some of the constant region, between the C_{H}1 and C_{H}2 domains, such that an interchain disulfide bond can be formed between two heavy chains.

As used herein, a “flexible linker” refers to any linker that is not predicted by one skilled in the art, according to its chemical structure, to be fixed in three-dimensional space. In certain embodiments, a peptide linker comprising three or more amino acids is a flexible linker.

The terms “variable region” and “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target The term “immunologically functional immunoglobulin fragment” refers to a polypeptide fragment comprising at least the variable domains of an immunoglobulin heavy chain and an immunoglobulin light chain. In certain embodiments, an immunologically functional immunoglobulin fragment is capable of binding to a ligand, preventing binding of the ligand to its receptor, and thereby interrupting a biological response resulting from ligand binding to the receptor. In certain embodiments, an immunologically functional immunoglobulin fragment is capable of binding to a receptor, preventing binding of the ligand to its receptor, and thereby interrupting a biological response resulting from ligand binding to the receptor. In certain embodiments, an immunologically functional immunoglobulin fragment is capable of binding a receptor and activating that receptor. In certain embodiments, an immunologically functional immunoglobulin fragment is capable of binding a receptor and inactivating that receptor.

The term “target” refers to a molecule or a portion of a molecule capable of being bound by a specific binding agent. In certain embodiments, a target may have one or more epitopes. In certain embodiments, a target is an antigen.

The term “epitope” refers to a portion of a molecule capable of being bound by a specific binding agent. Exemplary epitopes may comprise any polypeptide determinant capable of specific binding to an immunoglobulin and/or T-cell receptor. Exemplary epitope determinants include, but are not limited to, chemically active surface groupings of molecules, for example, but not limited to, amino acids, sugar side chains, phosphoryl groups, and sulfonyl groups. In certain embodiments, epitope determinants may have specific three dimensional structural characteristics, and/or specific charge characteristics. In certain embodiments, an epitope is a region of an antigen that is bound by an antibody. Epitopes may be contiguous or non-contiguous. In certain embodiments, epitopes may be mimetic in that they comprise a three dimensional structure that is similar to an epitope used to generate the antibody, yet comprise none or only some of the amino acid residues found in that epitope used to generate the antibody.

The term “inhibiting and/or neutralizing epitope” refers to an epitope, which when bound by a specific binding agent results in a decrease in a biological activity in vivo, in vitro, and/or in situ. In certain embodiments, a neutralizing epitope is located on or is associated with a biologically active region of a target.

The term “activating epitope” refers to an epitope, which when bound by a specific binding agent results in activation or maintenance of a biological activity in vivo, in vitro, and/or in situ. In certain embodiments, an activating epitope is located on or is associated with a biologically active region of a target.

The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “agent” is used herein to denote a chemical compound, a mixture of chemical compounds, a biological macromolecule, or an extract made from biological materials.

The term “isolated polynucleotide” as used herein means a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is linked to a polynucleotide which it is not linked to in nature, or (3) does not occur in nature as part of a larger sequence.

The term “operably linked” refers to components that are in a relationship permitting them to function in their intended manner. For example, in the context of a polynucleotide sequence, a control sequence may be “operably linked” to a coding sequence when the control sequence and coding sequence are in association with each other in such a way that expression of the coding sequence is achieved under conditions compatible with the functioning of the control sequence.

The term “control sequence” refers to polynucleotide sequences which may effect the expression and processing of coding sequences with which they are in association. The nature of such control sequences may differ depending upon the host organism. Certain exemplary control sequences for prokaryotes include, but are not limited to, promoters, ribosomal binding sites, and transcription termination sequences. Certain exemplary control sequences for eukaryotes include, but are not limited to, promoters, enhancers, and transcription termination sequences. In certain embodiments, “control sequences” can include leader sequences and/or fusion partner sequences.

The terms “isolated polypeptide” and “isolated peptide” refer to any polypeptide that (1) is free of at least some proteins with which it would normally be found, (2) is essentially free of other proteins from the same source, e.g., from the same species, (3) is expressed by a cell from a different species, or (4) does not occur in nature.

The terms “polypeptide,” “peptide,” and “protein” are used interchangeably herein and refer to a polymer of two or more amino acids joined to each other by peptide bonds or modified peptide bonds, i.e., peptide isosteres. The terms apply to amino acid polymers containing naturally occurring amino acids as well as amino acid polymers in which one or more amino acid residues is a non-naturally occurring amino acid or a chemical analogue of a naturally occurring amino acid. An amino acid polymer may contain one or more amino acid residues that has been modified by one or more natural processes, such as post-translational processing, and/or one or more amino acid residues that has been modified by one or more chemical modification techniques known in the art.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference for any purpose. Stereoisomers (e.g., D-amino acids) of the twenty conventional amino acids, unnatural amino acids such as α, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, a-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

A “fragment” of a reference polypeptide refers to a contiguous stretch of amino acids from any portion of the reference polypeptide. A fragment may be of any length that is less than the length of the reference polypeptide.

A “variant” of a reference polypeptide refers to a polypeptide having one or more amino acid substitutions, deletions, or insertions relative to the reference polypeptide. In certain embodiments, a variant of a reference polypeptide has an altered post-translational modification site (i.e., a glycosylation site). In certain embodiments, both a reference polypeptide and a variant of a reference polypeptide are specific binding agents. In certain embodiments, both a reference polypeptide and a variant of a reference polypeptide are antibodies.

Variants of a reference polypeptide include, but are not limited to, glycosylation variants. Glycosylation variants include variants in which the number and/or type of glycosylation sites have been altered as compared to the reference polypeptide. In certain embodiments, glycosylation variants of a reference polypeptide comprise a greater or a lesser number of N-linked glycosylation sites than the reference polypeptide. In certain embodiments, an N-linked glycosylation site is characterized by the sequence Asn-X-Ser or Asn-X-Thr, wherein the amino acid residue designated as X may be any amino acid residue except proline. In certain embodiments, glycosylation variants of a reference polypeptide comprise a rearrangement of N-linked carbohydrate chains wherein one or more N-linked glycosylation sites (typically those that are naturally occurring) are eliminated and one or more new N-linked sites are created.

Variants of a reference polypeptide include, but are not limited to, cysteine variants. In certain embodiments, cysteine variants include variants in which one or more cysteine residues of the reference polypeptide are replaced by one or more non-cysteine residues; and/or one or more non-cysteine residues of the reference polypeptide are replaced by one or more cysteine residues. Cysteine variants may be useful, in certain embodiments, when a particular polypeptide must be refolded into a biologically active conformation, e.g., after the isolation of insoluble inclusion bodies. In certain embodiments, cysteine variants of a reference polypeptide have fewer cysteine residues than the reference polypeptide. In certain embodiments, cysteine variants of a reference polypeptide have an even number of cysteines to minimize interactions resulting from unpaired cysteines. In certain embodiments, cysteine variants have more cysteine residues than the native protein.

In certain embodiments, conservative modifications to the heavy and light chains of a particular antibody (and corresponding modifications to the encoding nucleotides) will produce antibodies having functional and chemical characteristics similar to those of the original antibody. In contrast, in certain embodiments, substantial modifications in the functional and/or chemical characteristics of a particular antibody may be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

Certain desired amino acid substitutions (whether conservative or non-conservative) can be determined by those skilled in the art at the time such substitutions are desired. In certain embodiments, amino acid substitutions can be used to identify important residues of particular antibodies, such as those which may increase or decrease the affinity of the antibodies or the effector function of the antibodies.

In certain embodiments, the effects of an antibody may be evaluated by measuring a reduction in the amount of symptoms of the disease. In certain embodiments, the disease of interest may be caused by a pathogen. In certain embodiments, a disease may be established in an animal host by other methods including introduction of a substance (such as a carcinogen) and genetic manipulation. In certain embodiments, effects may be evaluated by detecting one or more adverse events in the animal host. The term “adverse event” includes, but is not limited to, an adverse reaction in an animal host that receives an antibody that is not present in an animal host that does not receive the antibody. In certain embodiments, adverse events include, but are not limited to, a fever, an immune response to an antibody, inflammation, and/or death of the animal host.

Various antibodies specific to an antigen may be produced in a number of ways. In certain embodiments, an antigen containing an epitope of interest may be introduced into an animal host (e.g., a mouse), thus producing antibodies specific to that epitope. In certain instances, antibodies specific to an epitope of interest may be obtained from biological samples taken from hosts that were naturally exposed to the epitope. In certain instances, introduction of human immunoglobulin (Ig) loci into mice in which the endogenous Ig genes have been inactivated offers the opportunity to obtain human monoclonal antibodies (MAbs).

A specific binding agent “substantially inhibits binding” of a ligand to a receptor when an excess of specific binding agent reduces the quantity of receptor bound to the ligand by at least about 20%, 40%, 60%, 80%, 85%, or more (as measured in an in vitro competitive binding assay). In certain embodiments, a specific binding agent is an antibody. In certain such embodiments, an antibody substantially inhibits binding of HGF to Met.

The term “cancer” includes, but is not limited to, solid tumors and hematologic malignancies. Exemplary cancers include, but are not limited to, breast cancer, colorectal cancer, gastric carcinoma, glioblastoma, glioma cancer, head and neck cancer, hereditary and sporadic papillary renal carcinoma, leukemia, lymphoma, Li-Fraumeni syndrome, malignant pleural mesothelioma, medulloblastoma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

The term “pharmaceutical agent or drug” as used herein refers to a chemical compound or composition capable of inducing a desired therapeutic effect when properly administered to a patient.

The term “modulator,” as used herein, is a compound that changes or alters the activity or function of a molecule. For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Certain exemplary activities and functions of a molecule include, but are not limited to, binding affinity, enzymatic activity, and signal transduction. Certain exemplary inhibitors include, but are not limited to, proteins, peptides, antibodies, peptibodies, carbohydrates or small organic molecules. Peptibodies are described in, e.g., U.S. Pat. No. 6,660,843 (corresponding to PCT Application No. WO01/83525).

As used herein, “substantially pure” means an object species is the predominant species present (i.e., on a molar basis it is more abundant than any other individual species in the composition). In certain embodiments, a substantially purified fraction is a composition wherein the object species comprises at least about 50 percent (on a molar basis) of all macromolecular species present. In certain embodiments, a substantially pure composition will comprise more than about 80%, 85%, 90%, 95%, or 99% of all macromolar species present in the composition. In certain embodiments, the object species is purified to essential homogeneity (contaminant species cannot be detected in the composition by conventional detection methods) wherein the composition consists essentially of a single macromolecular species.

The term “patient” includes human and animal subjects.

### Certain Inhibitors

In certain embodiments, an HGF-Met inhibitor is a specific binding agent to HGF. In certain embodiments, a specific binding agent to HGF is an antibody to HGF. In certain embodiments, an antibody to HGF is a fully human antibody to HGF. In certain embodiments, a fully human antibody to HGF is selected from 1.24.1, 1.29.1, 1.60.1, 1.61.3, 1.74.3, 1.75.1, 2.4.4, 2.12.1, 2.40.1, and 3.10.1. Antibodies 1.24.1, 1.29.1, 1.60.1, 1.61.3, 1.74.3, 1.75.1, 2.4.4, 2.12.1, 2.40.1, and 3.10.1 are described in U.S. Publication No. 2005/0118643. In certain embodiments, a fully human antibody to HGF is 2.12.1.

In certain embodiments, an antibody to HGF is L2G7 (Takeda-Galaxy Biotech).

In certain embodiments, an HGF-Met inhibitor is an HGF epitope. In certain embodiments, an HGF epitope may interfere with normal HGF-Met signaling.

In certain embodiments, an HGF-Met inhibitor is of the formula:

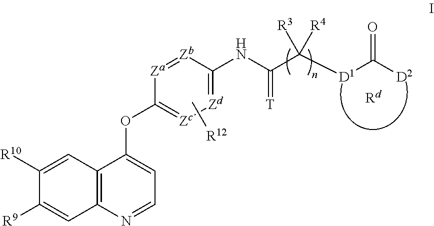

enantiomers, diastereomers, salts, solvates and N-Oxides thereof

wherein T is O or S;
wherein R³ and R⁴ is each independently selected from H, C₁₋₂-alkyl, phenyl, 5-6-membered heterocyclyl, phenyl-C₁₋₂-alkyl, 5-6-membered heterocyclyl-C₁₋₂-alkyl, C₃₋₆-cycloalkyl, and C₃₋₆-cycloalkyl-C₁₋₂-alkyl; alternatively R³ and R⁴, together with the atom they are attached to, form an optionally substituted 3-6 membered ring;
wherein R⁹ and R¹⁹ is independently selected from H, cyano, hydroxy, —C(═O)NR^{a}R^{5a}, 5-6 membered heterocyclyl, —NR^{a}C(═O)—R^{5a}, R^{5a}R^{a}N—O₂S—, R^{5a}O₂SR^{a}N—, R^{5a}R^{a}N—, alkyl, amino-C₁₋₆-alkyl, C₁₋₆-alkylamino-C₁₋₆-alkyl, alkoxy-C₁₋₆-alkyl, hydroxy, aryl-C₁₋₆-alkyl, heterocyclyl-C₁₋₆-alkyl, C₁₋₆-alkoxy, halo-C₁₋₆-alkoxy, C₁₋₆-alkylamino-C₁₋₆-alkoxy, aryl-C₁₋₆-alkoxy, 5-6-membered heterocyclyl, —C₁₋₆alkoxy, C₃₋₆-cycloalkyl-C₁₋₆-alkoxy, 5-6-membered heterocyclyl(hydroxyl-C₁₋₆-alkoxy), C₃₋₆-cycloalkyl(hydroxyl-C₁₋₆-alkoxy), phenyl(hydroxyl-C₁₋₆-alkoxy), C₁₋₆-alkoxy-C₁₋₆-alkoxy, phenyloxy-C₁₋₆-alkoxy, 5-6 membered heterocyclyloxy-C₁₋₆-alkoxy, C₃₋₆-cycloalkyloxy-C₁₋₆-alkoxy, phenyloxy, 5-6-membered heterocyclyloxy, and C₃₋₆-cycloalkyloxy;
wherein each of Z^{a}, Z^{b}, Z^{c} and Z^{d} is independently selected from N or CH; provided no more than 2 of Z^{a}, Z^{b}, Z^{c} and Z^{d} are N;
wherein n is 0, 1, 2 or 3;
wherein D¹ is selected from N or CR¹¹;
wherein D² is selected from NR¹³, O, or CHR¹¹; provided either D¹ is N or D² is NR¹³;
wherein ring R^{d} including

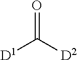

forms an optionally substituted optionally benzo-fused 4-7 membered heterocyclic moiety,
wherein R¹¹ is selected from H, halo, C₁₋₄-alkyl, C₁₋₄-haloalkyl, C₁₋₄-hydroxyalkyl, —NH₂, —OR¹², alkoxycarbonyl, —CO₂H, —CONR³R^{5a}, (C₁-C₃)alkylamino, di(C₁-C₆)alkylamino, (C₁-C₃)hydroxyalkylamino, (C₁-C₃)alkylamino-(C₁-C₃)alkylamino, C₁₋₃-alkoxy-C₁₋₃-alkyl, C₁₋₃-alkylamino-C₁₋₃-alkyl, C₁₋₃-alkylthio-C₁₋₃-alkyl, optionally substituted phenyl-C₁₋₃-alkyl, 5-6 membered heterocyclyl-C₁₋₃-alkyl, C₃₋₆-cycloalkyl-C₁₋₃-alkyl, optionally substituted phenyl, optionally substituted 5-6 membered heterocyclyl, and C₃₋₆-cycloalkyl;
wherein R^{a} is selected from H, alkyl, heterocyclyl, aryl, arylalkyl, heterocyclylalkyl, cycloalkyl, cycloalkylalkyl, alkenyl and alkynyl;
wherein R^{5a} is selected from H, alkyl, haloalkyl, arylalkyl, heterocyclylalkyl, cycloalkylalkyl, aryl, heterocyclyl, alkenyl, alkynyl and cycloalkyl;
wherein R¹² is selected from H, halo, C₁₋₂-alkyl and methoxy;
wherein R¹³ is selected from H, alkyl, haloalkyl, optionally substituted phenylalkyl, optionally substituted 5-10 membered heterocyclylalkyl, cycloalkylalkyl, optionally substituted phenyl or naphthyl, optionally substituted 5-10 membered heterocyclyl and cycloalkyl;
   and pharmaceutically acceptable salts thereof. Compounds of Formula I, including their structures and properties and methods for making and using them, are described in WO 2006/116713.

In certain embodiments, an HGF-Met inhibitor is selected from:
N-(4-(6,7-dimethoxyquinolin-4-yloxy)-3-fluorophenyl)-1-methyl-3-oxo-2-phenyl-5-(pyridin-4-yl)-2,3-dihydro-1H-pyrazole-4-carboxamide,
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(4-(6,7-dimethoxyquinolin-4-yloxy)-3-fluorophenyl)-5-((ethyl(methyl)amino)methyl)-1-methyl-3-oxo-2-phenyl-2,3-dihydro-1H-pyrazole-4-carboxamide;
N-(4-(6,7-dimethoxyquinolin-4-yloxy)-3-fluorophenyl)-5-((dimethylamino)methyl)-1-methyl-3-oxo-2-phenyl-2,3-dihydro-1H-pyrazole-4-carboxamide;
5-(aminomethyl)-N-(3-fluoro-4-(7-methoxyquinolin-4-yloxy)phenyl)-1-methyl-3-oxo-2-phenyl-2,3-dihydro-1H-pyrazole-4-carboxamide;
tert-butyl (4-((3-fluoro-4-(7-methoxyquinolin-4-yloxy)phenyl)carbamoyl)-1-methyl-3-oxo-2-phenyl-2,3-dihydro-1H-pyrazol-5-yl)methylcarbamate;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-5-methyl-3-oxo-2-phenyl-1-((tetrahydrofuran-2-yl)methyl)-2,3-dihydro-1H-pyrazole-4-carboxamide;
5-((ethyl(methyl)amino)methyl)-N-(3-fluoro-4-(7-methoxyquinolin-4-yloxy)phenyl)-1-methyl-3-oxo-2-phenyl-2,3-dihydro-1H-pyrazole-4-carboxamide;
2-benzyl-N-(5-(7-methoxyquinolin-4-yloxy)pyridin-2-yl)-1-methyl-3-oxo-5-(pyridin-4-yl)-2,3-dihydro-1H-pyrazole-4-carboxamide;
2-benzyl-N-(3-fluoro-4-(7-methoxyquinolin-4-yloxy)phenyl)-1-methyl-3-oxo-5-(pyridin-4-yl)-2,3-dihydro-1H-pyrazole-4-carboxamide;
(S)—N-(5-(7-methoxyquinolin-4-yloxy)pyridin-2-yl)-1-methyl-3-oxo-2-(1-phenylethyl)-5-(pyridin-4-yl)-2,3-dihydro-1H-pyrazole-4-carboxamide;
(S)—N-(3-fluoro-4-(7-methoxyquinolin-4-yloxy)phenyl)-1-methyl-3-oxo-2-(1-phenylethyl)-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-4-yl)-2,3-dihydro-1H-pyrazole-4-carboxamide;
N-(5-(6,7-dimethoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-2-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-2-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(tetrahydro-2H-pyran-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(tetrahydro-2H-pyran-4-yl)-2,3-dihydro-1H-pyrazole-4-carboxamide;
1-Methyl-N-(5-((7-(methyloxy)-4-quinolinyl)oxy)-2-pyridinyl)-5-(2-methyl-1,3-thiazol-4-yl)-3-oxo-2-phenyl-2,3-dihydro-1H-pyrazole-4-carboxamide;
N-(5-((6,7-bis(methyloxy)-4-quinolinyl)oxy)-2-pyridinyl)-1-methyl-5-(5-methyl-3-isoxazolyl)-3-oxo-2-phenyl-2,3-dihydro-1H-pyrazole-4-carboxamide;
1-methyl-5-(5-methyl-3-isoxazolyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(5-methyl-3-isoxazolyl)-3-oxo-2-phenyl-2,3-dihydro-1H-pyrazole-4-carboxamide;
1-methyl-N-(5-((7-(methyloxy)-4-quinolinyl)oxy)-2-pyridinyl)-3-oxo-2-phenyl-5-(2-pyrazinyl)-2,3-dihydro-1H-pyrazole-4-carboxamide;
N-(3-fluoro-4-((7-(methyloxy)-4-quinolinyl)oxy)phenyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-5-(2-methyl-1,3-thiazol-4-yl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(2-methyl-1,3-thiazol-4-yl)-3-oxo-2-phenyl-2,3-dihydro-1H-pyrazole-4-carboxamide;
N-(4-(6,7-dimethoxyquinolin-4-yloxy)-3-fluorophenyl)-N,1,5-trimethyl-3-oxo-2-phenyl-2,3-dihydro-1H-pyrazole-4-carboxamide;
2-(3-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(3-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
N-(4-(6,7-dimethoxyquinolin-4-yloxy)-3-fluorophenyl)-1,5-dimethyl-3-oxo-2-p-tolyl-2,3-dihydro-1H-pyrazole-4-carboxamide;
N-(4-(6,7-dimethoxyquinolin-4-yloxy)-3-fluorophenyl)-2-(4-fluorophenyl)-1,5-dimethyl-3-oxo-2,3-dihydro-1H-pyrazole-4-carboxamide;
N-(5-(6,7-dimethoxyquinolin-4-yloxy)pyridine-2-yl)-1,5-dimethyl-3-oxo-2-p-tolyl-2,3-dihydro-1H-pyrazole-4-carboxamide;
N-(5-(6,7-dimethoxyquinolin-4-yloxy)pyridin-2-yl)-2-(4-fluorophenyl)-1,5-dimethyl-3-oxo-2,3-dihydro-1H-pyrazole-4-carboxamide;
2-(3-chlorophenyl)-N-(5-(7-methoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1,5-dimethyl-3-oxo-2-p-tolyl-2,3-dihydro-1H-pyrazole-4-carboxamide;
2-(2-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
2-(2-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(2-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(3-fluoro-4-(7-methoxyquinolin-4-yloxy)phenyl)-2-(4-fluorophenyl)-1,5-dimethyl-3-oxo-2,3-dihydro-1H-pyrazole-4-carboxamide;
2-(3-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(6-(6,7-dimethoxyquinolin-4-yloxy)pyridin-3-yl)-1,5-dimethyl-3-oxo-2-phenyl-2,3-dihydro-1H-pyrazole-4-carboxamide;
N-(2-chloro-4-(6,7-dimethoxyquinolin-4-yloxy)phenyl)-1,5-dimethyl-3-oxo-2-phenyl-2,3-dihydro-1H-pyrazole-4-carboxamide;
2-benzyl-N-(4-(6,7-dimethoxyquinolin-4-yloxy)-3-fluorophenyl)-1,5-dimethyl-3-oxo-2,3-dihydro-1H-pyrazole-4-carboxamide;
2-benzyl-N-(5-(6,7-dimethoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1-methyl-3-oxo-2-phenyl-2,3-dihydro-1H-pyrazole-4-carboxamide;
N-(4-(6,7-dimethoxyquinolin-4-yloxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-3-oxo-1-(2-oxobutyl)-2-phenyl-2,3-dihydro-1H-pyrazole-4-carboxamide;
N-(3-fluoro-4-(7-methoxyquinolin-4-yloxy)phenyl)-5-methyl-1-(3-methyl-2-oxobutyl)-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-((2R,3R)-3-hydroxybutan-2-yl)-5-methyl-3-oxo-2-phenyl-2,3-dihydro-1H-pyrazole-4-carboxamide;
1-((2R,3R)-3-hydroxybutan-2-yl)-N-(5-(7-methoxyquinolin-4-yloxy)pyridin-2-yl)-5-methyl-3-oxo-2-phenyl-2,3-dihydro-1H-pyrazole-4-carboxamide;
(S)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(R)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(S)—N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-((3-methyl-2-oxooxazolidin-5-yl)methyl)-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-(methylamino)propyl)-5-methyl-3-oxo-2-phenyl-2,3-dihydro-1H-pyrazole-4-carboxamide;
1-(3-chloro-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylbutyl)-5-methyl-3-oxo-2-phenyl-2,3-dihydro-1H-pyrazole-4-carboxamide;
1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-morpholinopropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-(oxazolidin-5-ylmethyl)-3-oxo-2-phenyl-2,3-dihydro-1H-pyrazole-4-carboxamide;
(S)—N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
1-(3-amino-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylpropyl)-5-methyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
1-(3-(dimethylamino)-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxypropyl)-5-methyl-3-oxo-2-phenyl-2,3-dihydro-1H-pyrazole-4-carboxamide;
(R)—N-(4-(6,7-dimethoxyquinolin-4-yloxy)-3-fluorophenyl)-1-(2-hydroxypropyl)-5-methyl-3-oxo-2-phenyl-2,3-dihydro-1H-pyrazole-4-carboxamide;
1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
N-(5-(6,7-dimethoxyquinolin-4-yloxy)pyridin-2-yl)-1-(2-hydroxy-2-methylpropyl)-5-methyl-3-oxo-2-phenyl-2,3-dihydro-1H-pyrazole-4-carboxamide;
(R)-2-(3-chlorophenyl)-N-(3-fluoro-4-(7-methoxyquinolin-4-yloxy)phenyl)-1-(2-hydroxypropyl)-5-methyl-3-oxo-2,3-dihydro-1H-pyrazole-4-carboxamide;
(R)-2-(3-chlorophenyl)-1-(2-hydroxypropyl)-N-(5-(7-methoxyquinolin-4-yloxy)pyridin-2-yl)-5-methyl-3-oxo-2,3-dihydro-1H-pyrazole-4-carboxamide;
(R)—N-(3-fluoro-4-(7-methoxyquinolin-4-yloxy)phenyl)-2-(4-fluorophenyl)-1-(2-hydroxypropyl)-5-methyl-3-oxo-2,3-dihydro-1H-pyrazole-4-carboxamide
1-(2-hydroxy-2-methylpropyl)-N-(5-(1-oxo-7-methoxyquinolin-4-yloxy)pyridin-2-yl)-5-methyl-3-oxo-2-phenyl-2,3-dihydro-1H-pyrazole-4-carboxamide;
N-(3-Fluoro-4-(7-hydroxyquinolin-4-yloxy)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-N-(5-(7-hydroxyquinolin-4-yloxy)pyridin-2-yl)-5-methyl-3-oxo-2-phenyl-2,3-dihydro-1H-pyrazole-4-carboxamide;
N-(4-(6-Ethyl-7-methoxyquinolin-4-yloxy)-3-fluorophenyl)-1,5-dimethyl-3-oxo-2-phenyl-2,3-dihydro-1H-pyrazole-4-carboxamide;
N-(3-Fluoro-4-(7-methoxyquinolin-4-yloxy)phenyl)-1,5-dimethyl-3-oxo-2-phenyl-2,3-dihydro-1H-pyrazole-4-carboxamide;
N-(3-fluoro-4-(7-Methoxyquinolin-4-yloxy)phenyl)-1,2-dimethyl-3-oxo-5-phenyl-2,3-dihydro-1H-pyrazole-4-carboxamide;
N-(5-(7-Methoxyquinolin-4-yloxy)pyridin-2-yl)-1,2-dimethyl-3-oxo-5-phenyl-2,3-dihydro-1H-pyrazole-4-carboxamide;
N-(4-(6,7-Dimethoxyquinolin-4-yloxy)-3-fluorophenyl)-1,2-dimethyl-3-oxo-5-phenyl-2,3-dihydro-1H-pyrazole-4-carboxamide;
N-(5-(7-Methoxyquinolin-4-yloxy)pyridin-2-yl)-1,5-dimethyl-3-oxo-2-phenyl-2,3-dihydro-1H-pyrazole-4-carboxamide;
(R)-1-(2-Hydroxypropyl)-N-(5-(7-methoxyquinolin-4-yloxy)pyridin-2-yl)-2-methyl-3-oxo-5-phenyl-2,3-dihydro-1H-pyrazole-4-carboxamide;
(R)—N-(3-Fluoro-4-(7-methoxyquinolin-4-yloxy)phenyl)-1-(2-hydroxypropyl)-2-methyl-3-oxo-5-phenyl-2,3-dihydro-1H-pyrazole-4-carboxamide
(S)—N-(3-fluoro-4-(6-methoxyquinolin-4-yloxy)phenyl)-1-(2-hydroxypropyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide
1-(2-(1,3-dioxo-1,3-dihydro-2H-isoindol-2-yl)ethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-(phenylmethyl)-2,3-dihydro-1H-pyrazole-4-carboxamide
1-benzyl-N-(3-fluoro-4-(7-methoxyquinolin-4-yloxy)phenyl)-5-methyl-3-oxo-2-phenyl-2,3-dihydro-1H-pyrazole-4-carboxamide;
5-methyl-1-(2-(methyloxy)ethyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-(methyloxy)ethyl)-3-oxo-2-phenyl-2,3-dihydro-1H-pyrazole-4-carboxamide;
1-(2-hydroxyethyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
1-((2R)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
(S)-1-(2-(dimethylamino)propyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-(2-(1-pyrrolidinyl)ethyl)-2,3-dihydro-1H-pyrazole-4-carboxamide;
1-((2S)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-((2S)-2-fluoropropyl)-5-methyl-3-oxo-2-phenyl-2,3-dihydro-1H-pyrazole-4-carboxamide;
1-((2S)-2-(acetylamino)propyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-aminopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-azidopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxyethyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-1-((2R)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-1-(2-methylpropyl)-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-propyl-2,3-dihydro-1H-pyrazole-4-carboxamide;
N-(3-fluoro-4-((7-(methyloxy)-4-quinolinyl)oxy)phenyl)-5-methyl-3-oxo-1-(2-oxopropyl)-2-phenyl-2,3-dihydro-1H-pyrazole-4-carboxamide;
1-(2,3-dihydroxy-2-methylpropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxypropyl)-5-methyl-3-oxo-2-phenyl-2,3-dihydro-1H-pyrazole-4-carboxamide;
N-(4-((6,7-bis(methyloxy)-4-quinazolinyl)oxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-methyl-2-propen-1-yl)-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-3-oxo-1-(2-oxopropyl)-2-phenyl-2,3-dihydro-1H-pyrazole-4-carboxamide;
N-(4-((6,7-bis(methyloxy)-4-quinolinyl)oxy)-3-fluorophenyl)-1-(2,3-dihydroxy-2-methylpropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-1-(2-methyl-2-propen-1-yl)-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-(2-propen-1-yl)-2,3-dihydro-1H-pyrazole-4-carboxamide;
N-(4-((6,7-bis(methyloxy)-1-oxido-4-quinolinyl)oxy)-3-fluorophenyl)-5-methyl-3-oxo-2-phenyl-1-(2-propen-1-yl)-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-1-(phenylmethyl)-2,3-dihydro-1H-pyrazole-4-carboxamide;
4-(6,7-Dimethoxyquinolin-4-yloxy)-3-fluoro-N-(5-oxo-1-phenyl-2,5-dihydro-1H-pyrazol-3-yl)benzamide;
4-(6,7-Dimethoxyquinolin-4-yloxy)-N-((1,2-dimethyl-5-oxo-3-phenyl-2,5-dihydro-1H-pyrazol-4-yl)methyl)-3-fluorobenzamide;
4-(6,7-Dimethoxyquinolin-4-yloxy)-N-(2,3-dimethyl-5-oxo-1-phenyl-2,5-dihydro-1H-pyrazol-4-yl)-3-fluorobenzamide

4-(6,7-Dimethoxyquinolin-4-yloxy)-N-((2,3-dimethyl-5-oxo-1-phenyl-2,5-dihydro-1H-pyrazol-4-yl)methyl)-3-fluorobenzamide;
1-Benzyl-N-(5-(7-methoxyquinolin-4-yloxy)pyridin-2-yl)-2-oxo-1,2-dihydropyrazolo[1,5-a]pyridine-3-carboxamide;
4-((5-(6,7-Dimethoxyquinolin-4-yloxy)pyridin-2-ylamino)methyl)-1,5-dimethyl-2-phenyl-1,2-dihydropyrazol-3-one;
N-(3-fluoro-4-(2-(3-methyl-1,2,4-oxadiazol-5-yl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-((2R)-2-hydroxypropyl)-5-methyl-3-oxo-2-phenyl-2,3-dihydro-1H-pyrazole-4-carboxamide;
N-(3-fluoro-4-(7H-pyrrolo[2,3-d]pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(1H-pyrrolo[2,3-b]pyridin-4-yloxy)phenyl)-1-(2-hydroxy-2-methylpropyl)-5-methyl-3-oxo-2-phenyl-2,3-dihydro-1H-pyrazole-4-carboxamide;
Methyl(6-((4-(((1-(2-hydroxy-2-methylpropyl)-5-methyl-3-oxo-2-phenyl-2,3-dihydro-1H-pyrazol-4-yl)carbonyl)amino)phenyl)oxy)-1H-benzimidazol-2-yl)carbamate;
N-(4-(2-(azetidine-1-carbonyl)thieno[3,2-b]pyridin-7-yloxy)-3-fluorophenyl)-5-methyl-3-oxo-2-phenyl-1-propyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
N-(3-fluoro-4-(2-(1-methylpiperazine-4-carbonyl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(4-(2-(3-(dimethylamino)pyrrolidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N,N-dimethylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-(2-methoxyethyl)thieno[3,2-b]pyridine-2-carboxamide;
N-(4-(2-(azetidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
N-cyclopropyl-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide
7-(2-fluoro-4-(5-methyl-3-oxo-2-phenyl-1-propyl-2,3-dihydro-1H-pyrazole-4-carboxamido)phenoxy)thieno[3,2-b]pyridine-2-carboxamide;
N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(6-(pyrrolidine-1-carboxamido)pyrimidin-4-yloxy)phenyl)-5-methyl-3-oxo-2-phenyl-1-propyl-2,3-dihydro-1H-pyrazole-4-carboxamide;
N-(6-(4-(1,5-dimethyl-3-oxo-2-phenyl-2,3-dihydro-1H-pyrazole-4-carboxamido)-2-fluorophenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)piperidine-1-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)-4-methylpiperazine-1-carboxamide;
(R)—N-(4-(6-(3-(dimethylamino)pyrrolidine-1-carboxamido)pyrimidin-4-yloxy)-3-fluorophenyl)-5-methyl-3-oxo-2-phenyl-1-propyl-2,3-dihydro-1H-pyrazole-4-carboxamide;
(R)—N-(4-(6-aminopyrimidin-4-yloxy)-3-fluorophenyl)-1-(2-hydroxypropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1,5-dimethyl-3-oxo-2-phenyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)piperidine-1-carboxamide;
(R)—N-(4-(2-(3-(dimethylamino)pyrrolidine-1-carboxamido)pyridin-4-yloxy)-3-fluorophenyl)-1,5-dimethyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-5-methyl-3-oxo-2-phenyl-1-propyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide;
N-(4-(2-fluoro-4-(1-(2-hydroxy-2-methylpropyl)-5-methyl-3-oxo-2-phenyl-2,3-dihydro-1H-pyrazole-4-carboxamido)phenoxy)pyridin-2-yl)piperidine-1-carboxamide;
5-methyl-N-(4-((7-(methyloxy)-4-quinolinyl)methyl)phenyl)-3-oxo-2-phenyl-1-propyl-2,3-dihydro-1H-pyrazole-4-carboxamide;
N-(4-(hydroxy(7-methoxyquinolin-4-yl)methyl)phenyl)-5-methyl-3-oxo-2-phenyl-1-propyl-2,3-dihydro-1H-pyrazole-4-carboxamide;
1,5-dimethyl-N-(5-((7-(methyloxy)-4-quinolinyl)oxy)-2-pyrimidinyl)-3-oxo-2-phenyl-2,3-dihydro-1H-pyrazole-4-carboxamide;
5-methyl-N-(4-((7-(methyloxy)-4-quinolinyl)sulfinyl)phenyl)-3-oxo-2-phenyl-1-propyl-2,3-dihydro-1H-pyrazole-4-carboxamide;
1-(2-hydroxy-2-methylpropyl)-5-methyl-N-(4-((7-(methyloxy)-4-quinolinyl)thio)phenyl)-3-oxo-2-phenyl-2,3-dihydro-1H-pyrazole-4-carboxamide;
5-methyl-N-(4-((7-(methyloxy)-4-quinolinyl)thio)phenyl)-3-oxo-2-phenyl-1-propyl-2,3-dihydro-1H-pyrazole-4-carboxamide
5-methyl-N-(3-((7-(methyloxy)-4-quinolinyl)oxy)propyl)-3-oxo-2-phenyl-1-propyl-2,3-dihydro-1H-pyrazole-4-carboxamide;
5-methyl-N-(trans-4-((7-(methyloxy)-4-quinolinyl)oxy)cyclohexyl)-3-oxo-2-phenyl-1-propyl-2,3-dihydro-1H-pyrazole-4-carboxamide;
5-methyl-N-(cis-4-((7-(methyloxy)-4-quinolinyl)oxy)cyclohexyl)-3-oxo-2-phenyl-1-propyl-2,3-dihydro-1H-pyrazole-4-carboxamide;
1-(2-hydroxy-2-methylpropyl)-5-methyl-N-(trans-4-((7-(methyloxy)-4-quinolinyl)oxy)cyclohexyl)-3-oxo-2-phenyl-2,3-dihydro-1H-pyrazole-4-carboxamide;
5-methyl-N-(4-((7-(methyloxy)-4-quinolinyl)amino)phenyl)-3-oxo-2-phenyl-1-propyl-2,3-dihydro-1H-pyrazole-4-carboxamide;
5-methyl-N-(5-((7-(methyloxy)-4-quinolinyl)oxy)-2-pyrimidinyl)-3-oxo-2-phenyl-1-propyl-2,3-dihydro-1H-pyrazole-4-carboxamide;
N-(3-fluoro-4-((7-(methyloxy)-4-quinolinyl)amino)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-5-methyl-4-((7-((7-(methyloxy)-4-quinolinyl)oxy)-2,3-dihydro-4H-1,4-benzoxazin-4-yl)carbonyl)-2-phenyl-1,2-dihydro-3H-pyrazol-3-one;
1-(2-hydroxy-2-methylpropyl)-5-methyl-N-(4-((7-(methyloxy)-4-quinolinyl)amino)phenyl)⁻3-oxo-2-phenyl-2,3-dihydro-1H-pyrazole-4-carboxamide;
N-(4-(6,7-dimethoxyquinolin-4-yloxy)-3-fluorophenyl)-3-hydroxy-2-(1-oxoisoindolin-2-yl)propanamide;
N-(4-(6,7-dimethoxyquinolin-4-yloxy)-3-fluorophenyl)-2-(1-oxoisoindolin-2-yl)acetamide;
N-(4-(6,7-dimethoxyquinolin-4-yloxy)-3-fluorophenyl)-2-oxo-1,5-diphenyl-1,2-dihydropyridine-3-carboxamide;
N-(5-((6,7-bis(methyloxy)-4-quinolinyl)oxy)-2-pyridinyl)-6-oxo-1-(phenylmethyl)-1,1′,2′,3′,6,6′-hexahydro-3,4′-bipyridine-5-carboxamide;
N-(5-((6,7-bis(methyloxy)-4-quinolinyl)oxy)-2-pyridinyl)-6-oxo-1-(phenylmethyl)-1,6-dihydro-3,3′-bipyridine-5-carboxamide;
N-(5-((6,7-bis(methyloxy)-4-quinolinyl)oxy)-2-pyridinyl)-6′-oxo-1′-(phenylmethyl)-1′,6′-dihydro-2,3′-bipyridine-5′-carboxamide;
N-(5-((6,7-bis(methyloxy)-4-quinolinyl)oxy)-2-pyridinyl)-2-oxo-1-(phenylmethyl)-5-(2-thienyl)-1,2-dihydro-3-pyridinecarboxamide;
N-(5-((6,7-bis(methyloxy)-4-quinolinyl)oxy)-2-pyridinyl)-2-oxo-1-(phenylmethyl)-5-(2-pyrazinyl)-1,2-dihydro-3-pyridinecarboxamide;
N-(5-((6,7-bis(methyloxy)-4-quinolinyl)oxy)-2-pyridinyl)-5-methyl-2-oxo-1-(phenylmethyl)-1,2-dihydro-3-pyridinecarboxamide;
N-(4-((6,7-bis(methyloxy)-4-quinolinyl)oxy)-3-fluorophenyl)-5-bromo-1-(3-methylphenyl)-2-oxo-1,2-dihydro-3-pyridinecarboxamide;
N-(4-((6,7-bis(methyloxy)-4-quinolinyl)oxy)-3-fluorophenyl)-5-(1-methyl-1H-pyrazol-4-yl)-2-oxo-1-phenyl-1,2-dihydro-3-pyridinecarboxamide;
N-(3-fluoro-4-((6-(methyloxy)-7-((3-(4-morpholinyl)propyl)oxy)-4-quinolinyl)oxy)phenyl)-2-oxo-5-phenyl-1-(phenylmethyl)-1,2-dihydro-3-pyridinecarboxamide;
1,1-dimethylethyl 5-(((5-((6,7-bis(methyloxy)-4-quinolinyl)oxy)-2-pyridinyl)amino)carbonyl)-6-oxo-1-(phenylmethyl)-1,3′,6,6′-tetrahydro-3,4′-bipyridine-1′(2′H)-carboxylate;
N-(4-((6,7-bis(methyloxy)-4-quinolinyl)oxy)-3-fluorophenyl)-2-oxo-1-(phenylmethyl)-5-(2-pyrimidinyl)-1,2-dihydro-3-pyridinecarboxamide;
N-(4-((6,7-bis(methyloxy)-4-quinolinyl)oxy)-3-fluorophenyl)-2-oxo-1-phenyl-5-(1H-pyrazol-4-yl)-1,2-dihydro-3-pyridinecarboxamide;
1-benzyl-5-bromo-N-(2-chloro-4-(6,7-dimethoxyquinolin-4-yloxy)phenyl)-2-oxo-1,2-dihydropyridine-3-carboxamide;
N-(5-(7-methoxyquinolin-4-yloxy)pyridin-2-yl)-2-oxo-1-phenyl-5-(pyridin-3-yl)-1,2-dihydropyridine-3-carboxamide,
N-(5-(7-methoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(pyridin-3-yl)-1,2-dihydropyridine-3-carboxamide;
N-(5-(6,7-dimethoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(thiophen-2-yl)-1,2-dihydropyridine-3-carboxamide;
5-benzyl-N-(5-(6,7-dimethoxyquinolin-4-yloxy)pyridin-2-yl)-2-oxo-1-phenyl-1,2-dihydropyridine-3-carboxamide;
tert-butyl 4-(5-((5-(6,7-dimethoxyquinolin-4-yloxy)pyridin-2-yl)carbamoyl)-6-oxo-1-phenyl-1,6-dihydropyridin-3-yl)-5,6-dihydropyridine-1(2H)-carboxylate;
5-bromo-N-(2-chloro-4-(6,7-dimethoxyquinolin-4-yloxy)phenyl)-2-oxo-1-phenyl-1,2-dihydropyridine-3-carboxamide;
N-(5-(6,7-dimethoxyquinolin-4-yloxy)pyridin-2-yl)-4-(2-methoxyethylamino)-2-oxo-1-phenyl-1,2-dihydropyridine-3-carboxamide;
N-(5-(6,7-dimethoxyquinolin-4-yloxy)pyridin-2-yl)-2-oxo-1-phenyl-4-(tetrahydro-2H-pyran-4-ylamino)-1,2-dihydropyridine-3-carboxamide;
N-(5-(6,7-dimethoxyquinolin-4-yloxy)pyridin-2-yl)-2-oxo-1-phenyl-4-(phenylamino)-1,2-dihydropyridine-3-carboxamide;
N-(5-(6,7-dimethoxyquinolin-4-yloxy)pyridin-2-yl)-4-(4-methylpiperazin-1-yl)-2-oxo-1-phenyl-1,2-dihydropyridine-3-carboxamide,
N-(5-(6,7-dimethoxyquinolin-4-yloxy)pyridin-2-yl)-4-(methylamino)-2-oxo-1-phenyl-1,2-dihydropyridine-3-carboxamide;
N-(5-(6,7-dimethoxyquinolin-4-yloxy)pyridin-2-yl)-4-(dimethylamino)-2-oxo-1-phenyl-1,2-dihydropyridine-3-carboxamide;
4-(2-methoxyethylamino)-N-(5-(7-methoxyquinolin-4-yloxy)pyridin-2-yl)-2-oxo-1-phenyl-1,2-dihydropyridine-3-carboxamide;
N-(3-fluoro-4-(7-methoxyquinolin-4-yloxy)phenyl)-4-(2-methoxyethylamino)-2-oxo-1-phenyl-1,2-dihydropyridine-3-carboxamide;
N-(4-((6,7-bis(methyloxy)-4-quinolinyl)oxy)-3-fluorophenyl)-1-cyclopentyl-6-oxo-5-(2-oxo-1-pyrrolidinyl)-1,6-dihydro-3-pyridinecarboxamide,
1-benzyl-N-(5-(6,7-dimethoxyquinolin-4-yloxy)pyridin-2-yl)-4-(2-methoxyethylamino)-2-oxo-1,2-dihydropyridine-3-carboxamide;
1-benzyl-N-(5-(6,7-dimethoxyquinolin-4-yloxy)pyridin-2-yl)-4-(dimethylamino)-2-oxo-1,2-dihydropyridine-3-carboxamide;
1-benzyl-N-(5-(6,7-dimethoxyquinolin-4-yloxy)pyridin-2-yl)-4-(methylamino)-2-oxo-1,2-dihydropyridine-3-carboxamide;
1-benzyl-N-(5-(6,7-dimethoxyquinolin-4-yloxy)pyridin-2-yl)-2-oxo-4-(phenylamino)-1,2-dihydropyridine-3-carboxamide;
1-benzyl-N-(5-(6,7-dimethoxyquinolin-4-yloxy)pyridin-2-yl)-2-oxo-4-(pyridin-4-ylamino)-1,2-dihydropyridine-3-carboxamide;
1-benzyl-N-(5-(6,7-dimethoxyquinolin-4-yloxy)pyridin-2-yl)-4-(4-methylpiperazin-1-yl)-2-oxo-1,2-dihydropyridine-3-carboxamide;
1-benzyl-N-(5-(6,7-dimethoxyquinolin-4-yloxy)pyridin-2-yl)-2-oxo-4-(tetrahydro-2H-pyran-4-ylamino)-1,2-dihydropyridine-3-carboxamide;
1-benzyl-N-(5-(6,7-dimethoxyquinolin-4-yloxy)pyridin-2-yl)-2-oxo-4-(4-(trifluoromethyl)phenylamino)-1,2-dihydropyridine-3-carboxamide;
1-cyclopentyl-N-(4-(6,7-dimethoxyquinolin-4-yloxy)-3-fluorophenyl)-6-oxo-5-(2-oxopyrrolidin-1-yl)-1,6-dihydropyridine-3-carboxamide;
N-(3-fluoro-4-(2-(pyrrolidine-1-carboxamido)pyridin-4-yloxy)phenyl)-3-oxo-2-phenyl-2,3-dihydropyridazine-4-carboxamide;
6-((diethylamino)methyl)-N-(4-(6,7-dimethoxyquinolin-4-yloxy)-3-fluorophenyl)-3-oxo-2-phenyl-2,3-dihydropyridazine-4-carboxamide;
6-((dimethylamino)methyl)-N-(3-fluoro-4-(7-methoxyquinolin-4-yloxy)phenyl)-3-oxo-2-phenyl-2,3-dihydropyridazine-4-carboxamide;
N-(3-fluoro-4-(7-methoxyquinolin-4-yloxy)phenyl)-6-methyl-3-oxo-2-phenyl-2,3-dihydropyridazine-4-carboxamide;
N-(5-(6,7-dimethoxyquinolin-4-yloxy)pyridin-2-yl)-6-methyl-3-oxo-2-phenyl-2,3-dihydropyridazine-4-carboxamide;
2-benzyl-N-(5-(6,7-dimethoxyquinolin-4-yloxy)pyridin-2-yl)-6-methyl-3-oxo-2,3-dihydropyridazine-4-carboxamide;
N-(3-fluoro-4-(7-methoxyquinolin-4-yloxy)phenyl)-3-oxo-2-phenyl-2,3-dihydropyridazine-4-carboxamide;
N-(2-chloro-4-(6,7-dimethoxyquinolin-4-yloxy)phenyl)-6-methyl-3-oxo-2-phenyl-2,3-dihydropyridazine-4-carboxamide;
(R)—N-(4-(6,7-dimethoxyquinolin-4-yloxy)-3-fluorophenyl)-6-((3-(dimethylamino)pyrrolidin-1-yl)methyl)-3-oxo-2-phenyl-2,3-dihydropyridazine-4-carboxamide;
3-benzyl-N-(4-(6,7-dimethoxyquinolin-4-yloxy)-3-fluorophenyl)-2-oxoimidazolidine-1-carboxamide;
N-(4-(6,7-dimethoxyquinolin-4-yloxy)-3-fluorophenyl)-5-((dimethylamino)methyl)-2-oxo-3-phenyl-tetrahydropyrimidine-1(2H)-carboxamide;
N-(3-Fluoro-4-(7-methoxyquinolin-4-yloxy)phenyl)-3-oxo-4-phenylmorpholine-2-carboxamide;
N-(5-(7-methoxyquinolin-4-yloxy)pyridin-2-yl)-1-methyl-3-oxo-2-phenyl-2,3-dihydro-1H-pyrazole-4-carboxamide; and
N-(3-Fluoro-4-(7-methoxyquinolin-4-yloxy)phenyl)-3-oxo-4-phenylmorpholine-2-carboxamide.
   Those compounds, including their structures and properties and methods for making and using them, are described in WO 2006/116713.

In certain embodiments, an HGF-Met inhibitor is selected from: AMG208, AMG458, XL880 (Exelixis) (also called EXEL-2880, among others), a multi-kinase inhibitor that interferes with c-Met pathways, including a formulation for oral administration, and closely related c-Met inhibitors; XL184 (Exelixis), including formulations for oral administration, and closely related c-Met inhibitors; PF-2341066 (Pfizer) including formulations for oral administration, and closely related c-Met inhibitors; ARQ197 (ArQule) including formulations for oral administration, and closely related c-Met inhibitors; MK2461 (Merck) including formulations for oral administration, and closely related c-Met inhibitors; MP-470 (SuperGen) including formulations for oral administration, and closely related c-Met inhibitors; and Kirin Compound 1 and related compounds. The chemical name of Kirin Compound is N-[4-(6,7-dimethoxyquinolin-4-yloxy)-3-Fluorophenyl]-N-phenylactylthiourea. Kirin Compound 1 and related compounds are described in US Patent Publication 2004/0242603. As used herein the term Kirin Compound 1 includes pharmaceutically acceptable salts.

In certain embodiments, an HGF-Met inhibitor is AMG208. In certain embodiments, an HGF-Met inhibitor is AMG458. AMG208 and AMG458, including their structures and properties, and methods for making and using them, are described in WO 2006/116713.

In certain embodiments, an HGF-Met inhibitor is a specific binding agent to Met. In certain embodiments, a specific binding agent to Met is an antibody. In certain embodiments, an antibody to Met is OA-5d5 (Genentech) (also called One Armed 5d5, 5d5, MetMab, PRO143966, among others). Antibody OA-5d5, including its structure and properties, and methods for making and using it, is described in U.S. Publication No. 2007/0092520. Additional exemplary antibodies to Met and methods of making and using such antibodies are described in, e.g., U.S. Pat. Nos. 5,646,036 and 5,686,292. In certain embodiments, an antibody to Met is a fully human monoclonal antibody to Met.

In certain embodiments, an EGFR inhibitor is a specific binding agent to EGFR. In certain embodiments, a specific binding agent to EGFR is an antibody to EGFR. In certain embodiments, an antibody to EGFR is selected from panitumumab, ERBITUX™, cetuximab, EMD72000, TheraCIM hR3, and LICR 806. In certain embodiments, an antibody to EGFR is a fully human monoclonal antibody to EGFR. In certain embodiments a fully human monoclonal antibody to EGFR is panitumumab. Panitumumab is described in U.S. Pat. No. 6,235,883. Additional exemplary antibodies to EGFR and methods of making and using such antibodies are also described in U.S. Pat. No. 6,235,883.

One can engineer mouse strains deficient in mouse antibody production with large fragments of the human Ig loci in anticipation that such mice would produce human antibodies in the absence of mouse antibodies. Large human Ig fragments may preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains may yield high affinity fully human antibodies against any antigen of interest. Using the hybridoma technology, antigen-specific human MAbs with the desired specificity may be produced and selected. Certain exemplary methods are described in WO 98/24893, U.S. Pat. No. 5,545,807, EP 546073B1, and EP 546073A1.

In certain embodiments, one may use constant regions from species other than human along with the human variable region(s). In certain embodiments, one may use constant regions from human along with variable region(s) from species other than human.

### Certain Exemplary Antibody Structure

Naturally occurring antibody structural units typically comprise a tetramer. Each such tetramer typically is composed of two identical pairs of polypeptide chains, each pair having one full-length light chain (in certain embodiments, about 25 kDa) and one full-length heavy chain (in certain embodiments, about 50-70 kDa).

The amino-terminal portion of each chain typically includes a variable region (V_{H} in the heavy chain and V_{L} in the light chain) of about 100 to 110 or more amino acids that typically is responsible for antigen recognition. The carboxy-terminal portion of each chain typically defines a constant region (C_{H} domains in the heavy chain and C_{L} in the light chain) that may be responsible for effector function. Antibody effector functions include activation of complement and stimulation of opsonophagocytosis. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology Ch. 7 (Paul, W., ed., 2nd ed. Raven Press, N.Y. (1989)). The variable regions of each light/heavy chain pair typically form the antigen binding site.

The variable regions typically exhibit the same general structure of relatively conserved framework regions (FR) joined by three hypervariable regions, also called complementarity determining regions or CDRs. The CDRs from the heavy and light chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3, and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

As discussed in the “Certain Definitions” section above, there are several types of antibody fragments. Exemplary antibody fragments include, but are not limited to, Fab fragment, Fab′ fragment, F(ab′)₂ molecule, Fv molecule, scFv, maxibody, and Fc fragment.

In certain embodiments, functional domains, C_{H}1, C_{H}2, C_{H}3, and intervening sequences can be shuffled to create a different antibody constant region. For example, in certain embodiments, such hybrid constant regions can be optimized for half-life in serum, for assembly and folding of the antibody tetramer, and/or for improved effector function. In certain embodiments, modified antibody constant regions may be produced by introducing single point mutations into the amino acid sequence of the constant region and testing the resulting antibody for improved qualities, e.g., one or more of those listed above.

In certain embodiments, an antibody of one isotype is converted to a different isotype by isotype switching without losing its specificity for a particular target molecule. Methods of isotype switching include, but are not limited to, direct recombinant techniques (see e.g., U.S. Pat. No. 4,816,397) and cell-cell fusion techniques (see e.g., U.S. Pat. No. 5,916,771), among others. In certain embodiments, an antibody can be converted from one subclass to another subclass using techniques described above or otherwise known in the art without losing its specificity for a particular target molecule, including, but not limited to, conversion from an IgG2 subclass to an IgG1, IgG3, or IgG4 subclass.

### Bispecific or Bifunctional Antibodies

A bispecific or bifunctional antibody typically is an artificial hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992).

### Certain Preparation of Antibodies

In certain embodiments, antibodies can be expressed in cell lines other than hybridoma cell lines. In certain embodiments, sequences encoding particular antibodies, including chimeric antibodies, can be used for transformation of a suitable mammalian host cell. According to certain embodiments, transformation can be by any known method for introducing polynucleotides into a host cell, including, for example packaging the polynucleotide in a virus (or into a viral vector) and transducing a host cell with the virus or by transfecting a vector using procedures known in the art, as exemplified by U.S. Pat. Nos. 4,399,216; 4,912,040; 4,740,461; and 4,959,455.

In certain embodiments, an expression vector comprises one or more polynucleotide sequences discussed herein, including, but not limited to, polynucleotide sequences encoding one or more antibodies. In certain embodiments, a method of making a polypeptide comprising producing the polypeptide in a cell comprising any of the above expression vectors in conditions suitable to express the polynucleotide contained therein to produce the polypeptide is provided.

In certain embodiments, an expression vector expresses an antibody heavy chain. In certain embodiments, an expression vector expresses an antibody light chain. In certain embodiments, an expression vector expresses both an antibody heavy chain and an antibody light chain. In certain embodiments, a method of making an antibody comprising producing the antibody in a cell comprising at least one of expression vectors in conditions suitable to express the polynucleotides contained therein to produce the antibody is provided.

In certain embodiments, the transfection procedure used may depend upon the host to be transformed. Certain methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Certain mammalian cell lines available as hosts for expression are known in the art and include, but are not limited to, many immortalized cell lines available from the American Type Culture Collection (ATCC), including but not limited to Chinese hamster ovary (CHO) cells, E5 cells, HeLa cells, baby hamster kidney (BHK) cells, monkey kidney cells (COS), human hepatocellular carcinoma cells (e.g., Hep G2), NSO cells, SP20 cells, Per C6 cells, 293 cells, and a number of other cell lines. In certain embodiments, cell lines may be selected through determining which cell lines have high expression levels and produce antibodies with constitutive antigen binding properties.

In certain embodiments, the vectors that may be transfected into a host cell comprise control sequences that are operably linked to a polynucleotide encoding an antibody. In certain embodiments, control sequences facilitate expression of the linked polynucleotide, thus resulting in the production of the polypeptide encoded by the linked polynucleotide. In certain embodiments, the vector also comprises polynucleotide sequences that allow chromosome-independent replication in the host cell. Exemplary vectors include, but are not limited to, plasmids (e.g., BlueScript, puc, etc.), cosmids, and YACS.

### Certain Compositions

In certain embodiments, pharmaceutical compositions comprising an HGF-Met inhibitor and/or an EGFR inhibitor are provided. In certain embodiments, a pharmaceutical composition comprises an HGF-Met inhibitor and an EGFR inhibitor. In certain embodiments, a pharmaceutical composition comprises an HGF-Met inhibitor. In certain embodiments, a pharmaceutical composition comprises an EGFR inhibitor. In certain embodiments, a pharmaceutical composition comprises an HGF-Met inhibitor and an EGFR inhibitor with a pharmaceutically acceptable diluent, vehicle, carrier, solubilizer, emulsifier, preservative and/or adjuvant. In certain embodiments, a pharmaceutical composition comprises an HGF-Met inhibitor with a pharmaceutically acceptable diluent, vehicle, carrier, solubilizer, emulsifier, preservative and/or adjuvant. In certain embodiments, a pharmaceutical composition comprises an EGFR inhibitor with a pharmaceutically acceptable diluent, vehicle, carrier, solubilizer, emulsifier, preservative and/or adjuvant.

In certain embodiments, a pharmaceutical composition includes more than one different HGF-Met inhibitor and more than one different EGFR inhibitor. In certain embodiments, a pharmaceutical composition includes an HGF-Met inhibitor and more than one different EGFR inhibitor. In certain embodiments, a pharmaceutical composition includes an EGFR inhibitor and more than one different HGF-Met inhibitor. In certain embodiments, a pharmaceutical composition includes more than one different HGF-Met inhibitor. In certain embodiments, a pharmaceutical composition includes more than one different EGFR inhibitor.

In certain embodiments, a pharmaceutical composition comprises an HGF-Met inhibitor and an EGFR inhibitor and a therapeutically effective amount of at least one additional therapeutic agent, together with a pharmaceutically acceptable diluent, carrier, solubilizer, emulsifier, preservative and/or adjuvant. In certain embodiments, a pharmaceutical composition comprises an HGF-Met inhibitor and a therapeutically effective amount of at least one additional therapeutic agent, together with a pharmaceutically acceptable diluent, carrier, solubilizer, emulsifier, preservative and/or adjuvant. In certain embodiments, a pharmaceutical composition comprises an EGFR inhibitor and a therapeutically effective amount of at least one additional therapeutic agent, together with a pharmaceutically acceptable diluent, carrier, solubilizer, emulsifier, preservative and/or adjuvant.

In certain embodiments, materials for compositions are nontoxic to recipients at the dosages and concentrations employed.

In certain embodiments, the primary vehicle or carrier in a pharmaceutical composition is aqueous in nature. In certain embodiments, a suitable vehicle or carrier may be water for injection, physiological saline solution, or artificial cerebrospinal fluid, possibly supplemented with other materials common in compositions for parenteral administration. In certain embodiments, the vehicle or carrier is sterile. In certain embodiments, additional components are included. Exemplary additional components include, but are not limited to, fixed oils; polyethylene glycols; glycerin; propylene glycol and other synthetic solvents; antibacterial agents including, but not limited to, benzyl alcohol and methyl parabens; antioxidants including, but not limited to, ascorbic acid and sodium bisulfite; and chelating agents including, but not limited to ethylenediaminetetraacetic acid. In certain embodiments, neutral buffered saline or saline mixed with serum albumin are further exemplary vehicles. In certain embodiments, pharmaceutical compositions comprise Tris buffer of about pH 7.0-8.5, or acetate buffer above pH 5.4, which may further include sorbitol or a suitable substitute therefore.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18^{th} Edition, A. R. Gennaro, ed., Mack Publishing Company (1990).

In certain embodiments, an HGF-Met inhibitor is linked to a half-life extending vehicle known in the art. In certain embodiments, an EGFR inhibitor is linked to a half-life extending vehicle known in the art. In certain embodiments, a therapeutic molecule is linked to a half-life extending vehicle known in the art. Such vehicles include, but are not limited to, polyethylene glycol, and dextran. Such vehicles are described, e.g., in U.S. application Ser. No. 09/428,082 and published PCT Application No. WO 99/25044.

In certain embodiments, a composition comprising an HGF-Met inhibitor and an EGFR inhibitor, with or without at least one additional therapeutic agents, may be prepared for storage by mixing the selected composition having the desired degree of purity with optional formulation agents (Remington's Pharmaceutical Sciences, supra) in the form of an aqueous solution. In certain embodiments, a composition comprising an HGF-Met inhibitor, with or without at least one additional therapeutic agents, may be prepared for storage by mixing the selected composition having the desired degree of purity with optional formulation agents (Remington's Pharmaceutical Sciences, supra) in the form of an aqueous solution. In certain embodiments, a composition comprising an EGFR inhibitor, with or without at least one additional therapeutic agents, may be prepared for storage by mixing the selected composition having the desired degree of purity with optional formulation agents (Remington's Pharmaceutical Sciences, supra) in the form of an aqueous solution. In certain embodiments, a pharmaceutical composition is enclosed in a container. Exemplary containers include, but are not limited to, an ampoule, disposable syringe, and multiple dose vial made of glass or plastic.

In certain embodiments, a liquid pharmaceutical composition is lyophilized. Certain methods for lyophilizing liquid compositions are known to those skilled in the art. In certain embodiments, the composition is reconstituted with a sterile diluent just prior to use. Exemplary sterile diluents include, but are not limited to, Ringer's solution, distilled water, and sterile saline. In certain embodiments, the composition is administered to patients upon reconstitution using methods known those skilled in the art.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, liquid, lyophylized, or spray-dried compositions comprising an HGF-Met inhibitor and an EGFR inhibitor are prepared as aqueous or nonaqueous solutions or suspensions for subsequent administration to a patient. In certain embodiments, liquid, lyophylized, or spray-dried compositions comprising an HGF-Met inhibitor are prepared as aqueous or nonaqueous solutions or suspensions for subsequent administration to a patient. In certain embodiments, liquid, lyophylized, or spray-dried compositions comprising an EGFR inhibitor are prepared as aqueous or nonaqueous solutions or suspensions for subsequent administration to a patient.

In certain embodiments, a pharmaceutical composition may be administered by any suitable route. In certain embodiments, a pharmaceutical composition may be administered in the form of a pharmaceutical composition adapted to a certain route. In certain embodiments, a pharmaceutical composition may be administered orally, mucosally, topically, rectally, pulmonarily such as by inhalation spray, or parenterally, including intravascularly, intravenously, intraperitoneally, subcutaneously, intramuscularly, intrasternally, and using infusion techniques.

In certain embodiments, a pharmaceutical composition can be selected for parenteral delivery. In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition may be in the form of a pyrogen-free, parenterally acceptable aqueous solution comprising an HGF-Met inhibitor and an EGFR inhibitor, with or without additional therapeutic agents, in a pharmaceutically acceptable vehicle. In certain embodiments, when parenteral administration is contemplated, a therapeutic composition may be in the form of a pyrogen-free, parenterally acceptable aqueous solution comprising an HGF-Met inhibitor, with or without additional therapeutic agents, in a pharmaceutically acceptable vehicle. In certain embodiments, when parenteral administration is contemplated, a therapeutic composition may be in the form of a pyrogen-free, parenterally acceptable aqueous solution comprising an EGFR inhibitor, with or without additional therapeutic agents, in a pharmaceutically acceptable vehicle.

In certain embodiments, a vehicle for parenteral injection is sterile distilled water in which an HGF-Met inhibitor and an EGFR inhibitor, with or without at least one additional therapeutic agent, is formulated as a sterile, isotonic solution, properly preserved. In certain embodiments, a vehicle for parenteral injection is sterile distilled water in which an HGF-Met inhibitor, with or without at least one additional therapeutic agent, is formulated as a sterile, isotonic solution, properly preserved. In certain embodiments, a vehicle for parenteral injection is sterile distilled water in which an EGFR inhibitor, with or without at least one additional therapeutic agent, is formulated as a sterile, isotonic solution, properly preserved.

In certain embodiments, preparation of the composition can involve the formulation of the desired molecule with an agent, such as injectable microspheres, bio-erodible particles, polymeric compounds (such as polylactic acid or polyglycolic acid), beads or liposomes, that may provide for the controlled or sustained release of the product which may then be delivered via a depot injection. In certain embodiments, hyaluronic acid may also be used, and may have the effect of promoting sustained duration in the circulation. In certain embodiments, implantable drug delivery devices may be used to introduce the desired molecule.

In certain embodiments, a composition for parenteral administration is in the form of an aqueous or non-aqueous, sterile, isotonic solution or suspension. In certain embodiments, such a solution or suspension may be prepared from sterile powders or granules by using one or more vehicles or carriers, or by using other suitable dispersing or wetting agents or suspending agents. In certain embodiments, a suitable vehicle or carrier is selected from water, saline, and dextrose. In certain embodiments, a composition for parenteral administration may contain additional components, including but not limited to polyethylene glycol, propylene glycol, ethanol, corn oil, cottonseed oil, peanut oil, sesame oil, benzyl alcohol, sodium chloride, tragacanth gum, and/or various buffers. In certain embodiments, a composition for parenteral administration contains cyclodextrin, for example Captisol; a compound for cosolvent solubilization, for example propylene glycol; or a compound for micellar solubilization, for example Tween 80.

In certain embodiments, a composition for parenteral administration is a sterile solution or suspension in a non-toxic parenterally acceptable solvent, for example 1,3-butanediol. In certain embodiments, acceptable solvents include sterile, fixed oils, including any bland fixed oil, including synthetic mono- or diglycerides, and fatty acids such as oleic acid.

In certain embodiments, a pharmaceutical composition may be formulated for inhalation. In certain embodiments, an HGF-Met inhibitor and an EGFR inhibitor, with or without at least one additional therapeutic agent, may be formulated as a dry powder for inhalation. In certain embodiments, an HGF-Met inhibitor, with or without at least one additional therapeutic agent, may be formulated as a dry powder for inhalation. In certain embodiments, an EGFR inhibitor, with or without at least one additional therapeutic agent, may be formulated as a dry powder for inhalation. In certain embodiments, an inhalation solution comprising an HGF-Met inhibitor and an EGFR inhibitor, with or without at least one additional therapeutic agent, may be formulated with a propellant for aerosol delivery. In certain embodiments, an inhalation solution comprising an HGF-Met inhibitor, with or without at least one additional therapeutic agent, may be formulated with a propellant for aerosol delivery. In certain embodiments, an inhalation solution comprising an EGFR inhibitor, with or without at least one additional therapeutic agent, may be formulated with a propellant for aerosol delivery. In certain embodiments, solutions may be nebulized. Pulmonary administration is further described in PCT application no. PCT/US94/001875, which describes pulmonary delivery of chemically modified proteins.

In certain embodiments, it is contemplated that formulations may be administered orally. In certain embodiments, an HGF-Met inhibitor and an EGFR inhibitor, with or without at least one additional therapeutic agent, that is administered in this fashion may be formulated with or without those carriers customarily used in the compounding of solid dosage forms such as capsules and tablets. In certain embodiments, an HGF-Met inhibitor, with or without at least one additional therapeutic agent, that is administered in this fashion may be formulated with or without those carriers customarily used in the compounding of solid dosage forms such as capsules and tablets. In certain embodiments, an EGFR inhibitor, with or without at least one additional therapeutic agent, that is administered in this fashion may be formulated with or without those carriers customarily used in the compounding of solid dosage forms such as capsules and tablets.

In certain embodiments, a capsule may be designed to release the active portion of the formulation at the point in the gastrointestinal tract when bioavailability is maximized and pre-systemic degradation is minimized. In certain embodiments, at least one additional agent can be included to facilitate absorption of a an HGF-Met inhibitor, an EGFR inhibitor, and/or any additional therapeutic agents. In certain embodiments, diluents, flavorings, low melting point waxes, vegetable oils, lubricants, suspending agents, tablet disintegrating agents, and binders may also be employed.

In certain embodiments, a pharmaceutical composition may involve an HGF-Met inhibitor and an EGFR inhibitor, with or without at least one additional therapeutic agent, in a mixture with non-toxic excipients which are suitable for the manufacture of tablets. In certain embodiments, a pharmaceutical composition may involve an HGF-Met inhibitor, with or without at least one additional therapeutic agent, in a mixture with non-toxic excipients which are suitable for the manufacture of tablets. In certain embodiments, a pharmaceutical composition may involve an EGFR inhibitor, with or without at least one additional therapeutic agent, in a mixture with non-toxic excipients which are suitable for the manufacture of tablets. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc. In certain embodiments, suitable excipients include, but are not limited to, sucrose, powder, cellulose esters of alkanoic acids, cellulose alkyl esters, magnesium oxide, sodium and calcium salts of phosphoric and sulfuric acids, sodium alginate, polyvinylpyrrolidone, and/or polyvinyl alcohol. In certain embodiments, by dissolving the tablets in sterile water, or another appropriate vehicle, solutions may be prepared in unit-dose form.

In certain embodiments, a pharmaceutical composition is in the form of a dosage unit comprising an amount of an HGF-Met inhibitor and/or an amount of an EGFR inhibitor. Examples of such dosage units are tablets and capsules. In certain embodiments, a pharmaceutical composition comprises an amount of an HGF-Met inhibitor and an amount of an EGFR inhibitor. In certain embodiments, a pharmaceutical composition comprising an amount of an HGF-Met inhibitor and an amount of an EGFR inhibitor comprises the same amounts of an HGF-Met inhibitor and an EGFR inhibitor. In certain embodiments, a pharmaceutical composition comprising an amount of an HGF-Met inhibitor and an amount of an EGFR inhibitor comprises different amounts of an HGF-Met inhibitor and an EGFR inhibitor. In certain embodiments, a pharmaceutical composition comprises an amount of an HGF-Met inhibitor. In certain embodiments, a pharmaceutical composition comprises an amount of an EGFR inhibitor.

In certain embodiments, a pharmaceutical composition comprises an amount of an HGF-Met inhibitor from about 1 to 2000 mg. In certain embodiments, a pharmaceutical composition comprises an amount of an EGFR inhibitor from about 1 to 2000 mg. In certain embodiments, a pharmaceutical composition comprises an amount of an HGF-Met inhibitor from about 1 to 500 mg. In certain embodiments, a pharmaceutical composition comprises an amount of an EGFR inhibitor from about 1 to 500 mg. In certain embodiments, a pharmaceutical composition comprises an amount of an HGF-Met inhibitor from about 10 mg to 150 mg. In certain embodiments, a pharmaceutical composition comprises an amount of an EGFR inhibitor from about 10 mg to 150 mg. In certain embodiments, a pharmaceutical composition comprises an amount of an HGF-Met inhibitor from about 25 to 125 mg. In certain embodiments, a pharmaceutical composition comprises an amount of an EGFR inhibitor from about 25 to 125 mg. In certain embodiments, a pharmaceutical composition comprises an amount of an HGF-Met inhibitor selected from about 25 mg, about 50 mg, about 75 mg, about 100 mg, about 150 mg, about 250 mg, about 350 mg, and about 500 mg. In certain embodiments, a pharmaceutical composition comprises an amount of an EGFR inhibitor selected from about 25 mg, about 50 mg, about 75 mg, about 100 mg, about 150 mg, about 250 mg, about 350 mg, and about 500 mg.

Additional pharmaceutical compositions will be evident to those skilled in the art, including formulations involving an HGF-Met inhibitor and an EGFR inhibitor, with or without at least one additional therapeutic agents, in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations may include semipermeable polymer matrices in the form of shaped articles, e.g. films, or microcapsules. Sustained release matrices may include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions may also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

In certain embodiments, a pharmaceutical composition is sterile. In certain embodiments, sterilization is accomplished by filtration through sterile filtration membranes. Where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it may be stored in sterile vials as a solution, suspension, gel, emulsion, solid, or as a dehydrated or lyophilized powder. In certain embodiments, such formulations may be stored either in a ready-to-use form or in a form (e.g., lyophilized) that is reconstituted prior to administration.

### Certain Kits

In certain embodiments, a kit comprising an HGF-Met inhibitor and an EGFR inhibitor is provided. In certain embodiments, a kit is designed for medical use. In certain embodiments, a kit comprises an HGF-Met inhibitor and an EGFR inhibitor in a pharmaceutically acceptable composition. In certain embodiments, a kit comprises an HGF-Met inhibitor in a pharmaceutically acceptable composition. In certain embodiments, a kit comprises an EGFR inhibitor in a pharmaceutically acceptable composition. In certain embodiments, a composition is formulated for reconstitution in a diluent. In certain embodiments, a kit comprises one or more containers of sterile diluent.

In certain embodiments, an HGF-Met inhibitor and an EGFR inhibitor is in one or more containers. In certain embodiments, an HGF-Met inhibitor and an EGFR inhibitor are in the same container. In certain embodiments, an HGF-Met inhibitor and an EGFR inhibitor are in separate containers. In certain embodiments, a composition comprising an HGF-Met inhibitor and an EGFR inhibitor is contained in a vial under partial vacuum sealed by a septum. In certain embodiments, that composition is suitable for reconstitution to form a composition effective for parental administration. In certain embodiments, a composition comprising an HGF-Met inhibitor is contained in a vial under partial vacuum sealed by a septum. In certain embodiments, that composition is suitable for reconstitution to form a composition effective for parental administration. In certain embodiments, a composition comprising an EGFR inhibitor is contained in a vial under partial vacuum sealed by a septum. In certain embodiments, that composition is suitable for reconstitution to form a composition effective for parental administration.

In certain embodiments, a kit comprises at least one single-dose administration unit. In certain embodiments, a kit comprises both a first container having a composition comprising a dried HGF-Met inhibitor and EGFR inhibitor and a second container having an aqueous formulation of that composition. In certain embodiments, a kit comprises both a first container having a composition comprising a dried HGF-Met inhibitor and a second container having an aqueous formulation of that composition. In certain embodiments, a kit comprises both a first container having a composition comprising a dried EGFR inhibitor and a second container having an aqueous formulation of that composition. In certain embodiments, a kit comprises at least one single or multi-chambered pre-filled syringes (e.g., liquid syringes and lyosyringes). In certain embodiments, the at least one single or multi-chambered pre-filled syringe is preloaded.

In certain embodiments, a kit comprises, integrally thereto or as one or more separate documents, information pertaining to the contents of the kit or the use an HGF-Met inhibitor and an EGFR inhibitor.

### Certain Therapeutic Uses

In certain embodiments, HGF binds Met to induce Met phosphorylation. In certain embodiments, normal HGF-induced Met phosphorylation results in HGF-Met activity. In certain embodiments, normal HGF-Met activity regulates a variety of cellular processes. In certain embodiments, aberrant HGF-Met activity correlates with certain cancers. Therefore, in certain embodiments, modulating HGF-Met activity may be therapeutically useful.

In certain embodiments, normal EGFR activity regulates a variety of cellular process. In certain embodiments, aberrant EGFR activity correlates with certain cancers. Therefore, in certain instances, modulating EGFR activity may be therapeutically useful. Exemplary cancers include, but are not limited to, breast cancer, colorectal cancer, gastric carcinoma, glioblastoma, glioma cancer, head and neck cancer, hereditary and sporadic papillary renal carcinoma, leukemia, lymphoma, L1-Fraumeni syndrome, malignant pleural mesothelioma, medulloblastoma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

In certain embodiments, a cancer is resistant to an HGF-Met inhibitor. In certain embodiments, a resistant cancer expresses EGFRvIII. In certain embodiments, methods are provided of treating a resistant cancer comprising administering a therapeutically effective amount of an HGF-Met inhibitor and an EGFR inhibitor. In certain embodiments, methods are provided of treating a resistant cancer comprising administering a therapeutically effective amount of a specific binding agent to HGF and an EGFR inhibitor. In certain embodiments, methods are provided of treating a resistant cancer comprising administering a therapeutically effective amount of an antibody to HGF and an EGFR inhibitor. In certain embodiments, methods are provided of treating a resistant cancer comprising administering a therapeutically effective amount of a fully human antibody to HGF and an EGFR inhibitor. In certain embodiments, methods are provided of treating a resistant cancer comprising administering a therapeutically effective amount of 2.12.1 and an EGFR inhibitor.

In certain embodiments, methods are provided of treating a resistant cancer comprising administering a therapeutically effective amount of an HGF-Met inhibitor and a specific binding agent to EGFR. In certain embodiments, methods are provided of treating a resistant cancer comprising administering a therapeutically effective amount of an HGF-Met inhibitor and an antibody to EGFR. In certain embodiments, methods are provided of treating a resistant cancer comprising administering a therapeutically effective amount of an HGF-Met inhibitor and a fully human antibody to EGFR. In certain embodiments, methods are provided of treating a resistant cancer comprising administering a therapeutically effective amount of an HGF-Met inhibitor and panitumumab.

In certain embodiments, methods are provided of treating a resistant cancer comprising administering a therapeutically effective amount of a specific binding agent to HGF and a specific binding agent to EGFR. In certain embodiments, methods are provided of treating a resistant cancer comprising administering a therapeutically effective amount of an antibody to HGF and an antibody to EGFR. In certain embodiments, methods are provided of treating a resistant cancer comprising administering a therapeutically effective amount of a fully human antibody to HGF and a fully human antibody to EGFR. In certain embodiments, methods are provided of treating a resistant cancer comprising administering a therapeutically effective amount of 2.12.1 and panitumumab.

In certain embodiments, methods are provided of treating a resistant cancer comprising administering a therapeutically effective amount of an HGF-Met inhibitor and an EGFR inhibitor and another therapeutic agent.

In certain embodiments, methods are provided of treating or preventing glioblastoma comprising administering a therapeutically effective amount of an HGF-Met inhibitor and an EGFR inhibitor. In certain embodiments, methods are provided of treating or preventing glioblastoma comprising administering a therapeutically effective amount of an HGF-Met inhibitor and an EGFR inhibitor and another therapeutic agent.

In certain embodiments, the administration of a therapeutically effective amount of an HGF-Met inhibitor and an EGFR inhibitor comprises administering an HGF-Met inhibitor and an EGFR inhibitor concurrently. In certain embodiments, the administration of a therapeutically effective amount of an HGF-Met inhibitor and an EGFR inhibitor comprises administering an HGF-Met inhibitor prior to an EGFR inhibitor. In certain embodiments, the administration of a therapeutically effective amount of an HGF-Met inhibitor and an EGFR inhibitor comprises administering an HGF-Met inhibitor subsequent to an EGFR inhibitor.

In certain embodiments, an HGF-Met inhibitor and an EGFR inhibitor are administered prior to the administration of at least one other therapeutic agent. In certain embodiments, an HGF-Met inhibitor and an EGFR inhibitor are administered concurrent with the administration of at least one other therapeutic agent. In certain embodiments, an HGF-Met inhibitor and an EGFR inhibitor are administered subsequent to the administration of at least one other therapeutic agent. Therapeutic agents, include, but are not limited to, at least one other cancer therapy agent. Exemplary cancer therapy agents include, but are not limited to, chemotherapy and radiation therapy.

Exemplary chemotherapy agents include, but are not limited to antineoplastic agents. Antineoplastic agents include, but are not limited to, antibiotic-type agents, alklylating agents, antimetabolite agents, hormonal agents, immunological agents, interferon-type agents, and miscellaneous agents.

In certain embodiments, an antineoplastic agent is an antimetabolite agent. Antimetabolite antineoplastic agents include, but are not limited to: 5-FU, fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

In certain embodiments, an antineoplastic agent is an alkylating-type agent. Alkylating-type antineoplastic agents include, but are not limited to: Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

In certain embodiments, an antineoplastic agent is an antibiotic-type antineoplastic agent. Suitable antibiotic-type antineoplastic agents include, but are not limited to: Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

Additional anti-neoplastic agent include, but are not limited to: tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

Additional anti-neoplastic agents include, but are not limited to: acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, BAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-N1, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburicase, rhenium Re 186 etidronate, RII retinamide, rituximab, romurtide, samarium (153 Sm) lexidronam, sargramostim, sizofuran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

In certain embodiments, an HGF-Met inhibitor and an EGFR inhibitor may be used with radiation. In certain embodiments, an HGF-Met inhibitor and an EGFR inhibitor may be used with agents used for hormonal therapy. Agents used for hormonal therapy include, but are not limited to, agents used for treatment of breast and prostate cancer, including aromatase inhibitors (e.g. Arimidex (chemical name: anastrozole), Aromasin (chemical name: exemestane), and Femara (chemical name: letrozole)); Serms (selective estrogen-receptor modulators) such as tamoxifen; and ERDs (estrogen-receptor downregulators), e.g. Faslodex (chemical name: fulvestrant).

Exemplary cancer therapies also include, but are not limited to, targeted therapies. Examples of targeted therapies include, but are not limited to, use of therapeutic antibodies. Exemplary therapeutic antibodies, include, but are not limited to, mouse, mouse-human chimeric, CDR-grafted, humanized and fully human antibodies, and synthetic antibodies, including, but not limited to, those selected by screening antibody libraries. Exemplary antibodies include, but are not limited to, those which bind to cell surface proteins Her2, CDC20, CDC33, mucin-like glycoprotein, and epidermal growth factor receptor (EGFR) present on tumor cells, and optionally induce a cytostatic and/or cytotoxic effect on tumor cells displaying these proteins.

In certain embodiments, cancer therapy agents are anti-angiogenic agents which decrease angiogenesis. In certain embodiments, cancer therapy agents are angiogenesis inhibitors.

In certain embodiments, an HGF-Met inhibitor and an EGFR inhibitor may be administered prophylactically to prevent or mitigate the onset of bone loss by metastatic cancer. In certain embodiments, an HGF-Met inhibitor and an EGFR inhibitor may be administered for the treatment of an existing condition of bone loss due to metastasis.

In certain embodiments, in view of the condition and the desired level of treatment, two, three, or more agents in addition to an HGF-Met inhibitor and an EGFR inhibitor may be administered. In certain embodiments, such agents may be provided together by inclusion in the same formulation. In certain embodiments, such agents and an HGF-Met inhibitor and an EGFR inhibitor may be provided together by inclusion in the same formulation. In certain embodiments, such agents and an HGF-Met inhibitor may be provided together by inclusion in the same formulation. In certain embodiments, such agents and an EGFR inhibitor may be provided together by inclusion in the same formulation. In certain embodiments, such agents may be formulated separately and provided together by inclusion in a treatment kit. In certain embodiments, such agents may be provided separately. In certain embodiments, when administered by gene therapy, the genes encoding protein agents and/or an HGF-Met inhibitor and/or an EGFR inhibitor may be included in the same vector. In certain embodiments, the genes encoding protein agents and/or an HGF-Met inhibitor and/or an EGFR inhibitor may be under the control of the same promoter region. In certain embodiments, the genes encoding protein agents and/or an HGF-Met inhibitor and/or an EGFR inhibitor may be in separate vectors.

It is understood that the response by individual patients to the aforementioned medications or combination therapies may vary, and an appropriate efficacious combination of drugs for each patient may be determined by his or her physician.

In certain embodiments, therapies comprising an HGF-Met inhibitor and an EGFR inhibitor and at least one serine protease inhibitor, and methods of treatment using such therapies are provided. In certain embodiments, a therapy comprises an HGF-Met inhibitor and an EGFR inhibitor, a serine protease inhibitor, and at least one additional agent described herein.

In certain instances, a disturbance of the protease/protease inhibitor balance can lead to protease-mediated tissue destruction, including, but not limited to, tumor invasion of normal tissue leading to metastasis.

In certain embodiments, the effective amount of an HGF-Met inhibitor and an EGFR inhibitor, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an HGF-Met inhibitor and an EGFR inhibitor, with or without at least one additional therapeutic agent, is being used, the route of administration, and the size (body weight, height, body surface and/or organ size) and/or condition (the age, physical condition, and/or general health) of the patient. In certain embodiments, the clinician will consider the severity and history of the disease for which an HGF-Met inhibitor and an EGFR inhibitor, with or without at least one additional therapeutic agent, is being used. In certain embodiments, the clinician may titer the dosage and modify the route of administration to obtain the optimal therapeutic effect.

In certain embodiments, a therapeutically effective dose of an HGF-Met inhibitor and an EGFR inhibitor comprises an amount of an HGF-Met inhibitor and an amount of an EGFR inhibitor. In certain embodiments, the amount of an HGF-Met inhibitor and the amount of an EGFR inhibitor in a therapeutically effective dose are the same. In certain embodiments, the amount of an HGF-Met inhibitor and the amount of an EGFR inhibitor in a therapeutically effective dose are different. In certain embodiments, a therapeutically effective dose of an HGF-Met inhibitor and an EGFR inhibitor comprises an amount of an HGF-Met inhibitor. In certain embodiments, a therapeutically effective dose of an HGF-Met inhibitor and an EGFR inhibitor comprises an amount of an EGFR inhibitor.

In certain embodiments, a therapeutically effective dose of an HGF-Met inhibitor and an EGFR inhibitor comprises an amount of an HGF-Met inhibitor that ranges from about 0.01 mg/kg to about 500 mg/kg, from about 0.01 mg/kg to about 50 mg/kg, or from about 0.01 mg/kg to about 30 mg/kg. In certain embodiments, a therapeutically effective dose of an HGF-Met inhibitor and an EGFR inhibitor comprises an amount of an EGFR inhibitor that ranges from about 0.01 mg/kg to about 500 mg/kg, from about 0.01 mg/kg to about 50 mg/kg, or from about 0.01 mg/kg to about 30 mg/kg.

In certain embodiments, a therapeutically effective dose of an HGF-Met inhibitor and an EGFR inhibitor comprises an amount of an antibody to HGF that ranges from about 0.5 mg/kg to about 30 mg/kg, administered weekly; about 2 mg/kg to about 20 mg/kg, administered weekly; about 1 mg/kg to about 20 mg/kg, administered every two weeks; about 3 mg/kg to about 20 mg/kg, administered every two weeks; or about 10 mg/kg to about 20 mg/kg, administered every two weeks. In certain embodiments, a therapeutically effective dose of an HGF-Met inhibitor and an EGFR inhibitor comprises an amount of an antibody to EGFR that ranges from about 0.5 mg/kg to about 10 mg/kg, administered weekly; about 2 mg/kg to about 3 mg/kg, administered weekly; about 2 mg/kg, administered weekly; about 1 mg/kg to about 15 mg/kg, administered every two weeks; about 3 mg/kg to about 10 mg/kg, administered every two weeks; about 6 mg/kg, administered every two weeks; about 2 mg/kg to about 30 mg/kg, administered every three weeks; about 5 mg/kg to about 15 mg/kg, administered every three weeks; or about 9 mg/kg, administered every three weeks.

In certain embodiments, a therapeutically effective dose of an HGF-Met inhibitor and an EGFR inhibitor comprises a dose of 10 mg/kg of an antibody to HGF administered every two weeks. In certain embodiments, a therapeutically effective dose of an HGF-Met inhibitor and an EGFR inhibitor comprises a dose of 6 mg/kg of an antibody to EGFR administered every two weeks. In certain embodiments, a therapeutically effective dose of an HGF-Met inhibitor and an EGFR inhibitor comprises a dose of 10 mg/kg of an antibody to HGF and a dose of 6 mg/kg of an antibody to EGFR administered every two weeks. In certain embodiments with that dosage of antibodies and frequency of administration, for each administration, the administration of the antibody to EGFR will be administered prior to the administration of the antibody to HGF. In certain embodiments with that dosage of antibodies and frequency of administration, for each administration, the administration of the antibody to EGFR will be administered after the administration of the antibody to HGF. In certain embodiments with that dosage of antibodies and frequency of administration, for each administration, the administration of the antibody to EGFR will be administered at the same time as the administration of the antibody to HGF.

In certain embodiments, the frequency of dosing will take into account the pharmacokinetic parameters of an HGF-Met inhibitor, an EGFR inhibitor and/or any additional therapeutic agents in the formulation used. In certain embodiments, the clinician may administer a therapeutically effective dose of an HGF-Met inhibitor and an EGFR inhibitor until the desired effect is achieved. In certain embodiments, a therapeutically effective dose of an HGF-Met inhibitor and an EGFR inhibitor may be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them.

In certain embodiments, a therapeutically effective dose of an HGF-Met inhibitor and an EGFR inhibitor used for treatment comprises an amount of an HGF-Met inhibitor that increases over the course of a patient treatment. In certain embodiments, a therapeutically effective dose of an HGF-Met inhibitor and an EGFR inhibitor used for treatment comprises an amount of an EGFR inhibitor that increases over the course of a patient treatment. In certain embodiments, a therapeutically effective dose of an HGF-Met inhibitor and an EGFR inhibitor used for treatment comprises an amount of an HGF-Met inhibitor that decreases over the course of a patient treatment. In certain embodiments, a therapeutically effective dose of an HGF-Met inhibitor and an EGFR inhibitor used for treatment comprises an amount of an EGFR inhibitor that decreases over the course of a patient treatment In certain embodiments, the dosing regimen includes an initial administration of a therapeutically effective dose of an HGF-Met inhibitor and an EGFR inhibitor, with or without at least one additional therapeutic agent, on days 1, 7, 14, and 21 of a treatment period. In certain embodiments, the dosing regimen includes an initial administration of a therapeutically effective dose of an HGF-Met inhibitor and an EGFR inhibitor, with or without at least one additional therapeutic agent, on days 1, 2, 3, 4, 5, 6, and 7 of a week in a treatment period. In certain embodiments, the dosing regimen includes an initial administration of a therapeutically effective dose of an HGF-Met inhibitor and an EGFR inhibitor, with or without at least one additional therapeutic agent, on days 1, 3, 5, and 7 of a week in a treatment period. In certain embodiments, the dosing regimen includes an initial administration of a therapeutically effective dose of an HGF-Met inhibitor and an EGFR inhibitor, with or without at least one additional therapeutic agent, on days 1 and 3 of a week in a treatment period. In certain embodiments, the dosing regimen includes an initial administration of a therapeutically effective dose of an HGF-Met inhibitor and an EGFR inhibitor, with or without at least one additional therapeutic agent, on day 1 of a week in a treatment period. In certain embodiments, the treatment period comprises 1 week, 2 weeks, 3 weeks, one month, 3 months, 6 months, one year, or more. In certain embodiments, treatment periods are subsequent or separated from each other by one day, one week, 2 weeks, one month, 3 months, 6 months, one year, or more. In certain embodiments, the dosing regimen includes an initial administration of a therapeutically effective dose of an HGF-Met inhibitor and an EGFR inhibitor, with or without at least one additional therapeutic agent, on day 1 of a treatment period that comprises 1 week. In certain embodiments, the dosing regimen includes an initial administration of a therapeutically effective dose of an HGF-Met inhibitor and an EGFR inhibitor, with or without at least one additional therapeutic agent, on day 1 of a treatment period that comprises 2 weeks. In certain embodiments, the dosing regimen includes an initial administration of a therapeutically effective dose of an HGF-Met inhibitor and an EGFR inhibitor, with or without at least one additional therapeutic agent, on day 1 of a treatment period that comprises 3 weeks.

In certain embodiments, the same therapeutically effective dose of an HGF-Met inhibitor and an EGFR inhibitor is administered at each dosing over the course of a treatment period. In certain embodiments, different therapeutically effective doses of an HGF-Met inhibitor and an EGFR inhibitor are administered at each dosing over the course of a treatment period. In certain embodiments, the same therapeutically effective dose of an HGF-Met inhibitor and an EGFR inhibitor is administered at certain dosings over the course of a treatment period and different therapeutically effective doses are administered at certain other dosings.

In certain embodiments, the initial therapeutically effective dose of an HGF-Met inhibitor and an EGFR inhibitor comprises an amount of an HGF-Met inhibitor in a lower dosing range, for example, from 0.1 μg/kg up to 20 mg/kg, with subsequent doses comprising an amount of an HGF-Met inhibitor in an upper dosing range, for example, from 20 mg/kg up to 100 mg/kg. In certain embodiments, the initial therapeutically effective dose of an HGF-Met inhibitor and an EGFR inhibitor comprises an amount of an EGFR inhibitor in a lower dosing range, for example, from 0.1 μg/kg up to 20 mg/kg, with subsequent doses comprising an amount of an EGFR inhibitor in an upper dosing range, for example, from 20 mg/kg up to 100 mg/kg. In certain embodiments, the initial therapeutically effective dose of an HGF-Met inhibitor and an EGFR inhibitor comprises an amount of an HGF-Met inhibitor in the upper dosing range, for example, from 20 mg/kg up to 100 mg/kg, with subsequent doses in a lower dosing range, for example, from 0.1 μg/kg up to 20 mg/kg. In certain embodiments, the initial therapeutically effective dose of an HGF-Met inhibitor and an EGFR inhibitor comprises a dose of an EGFR inhibitor in the upper dosing range, for example, from 20 mg/kg up to 100 mg/kg, with subsequent doses in a lower dosing range, for example, from 0.1 μg/kg up to 20 mg/kg. Those ranges and any ranges discussed in this application include the endpoints and all values between the endpoints.

In certain embodiments, the initial therapeutically effective dose an HGF-Met inhibitor and an EGFR inhibitor is administered as a “loading dose.” “Loading dose” refers to an initial dose of an HGF-Met inhibitor and an EGFR inhibitor that is administered to a patient, where the dose administered of the HGF-Met inhibitor and an EGFR inhibitor comprises an amount of an HGF-Met inhibitor and/or an amount of an EGFR inhibitor that that falls within a higher dosing range, for example, 20 mg/kg up to 100 mg/kg. In certain embodiments, the loading dose is administered as a single administration, for example, including, but not limited to, a single infusion administered intravenously. In certain embodiments, the loading dose is administered as multiple administrations, for example, including, but not limited to, multiple infusions administered intravenously. In certain embodiments, the loading dose is administered over a 24-hour period. In certain embodiments, the loading dose is administered over a period of from 18 to 24 hours. In certain embodiments, the loading dose is administered over a period of from 12 to 18 hours. In certain embodiments, the loading dose is administered over a period of from 6 to 12 hours. In certain embodiments, the loading dose is administered over a period of from 0 to 6 hours.

In certain embodiments, after administration of the loading dose, the patient is administered one or more additional therapeutically effective doses of an HGF-Met inhibitor and an EGFR inhibitor. In certain such embodiments, subsequent therapeutically effective doses of an HGF-Met inhibitor and an EGFR inhibitor are administered according to a weekly dosing schedule, for example, but not limited to, once every two weeks, once every three weeks, or once every four weeks. In certain such embodiments, the subsequent therapeutically effective doses comprise a dose of an HGF-Met inhibitor and/or a dose of an EGFR inhibitor that falls within a lower dosing range, for example, 0.1 mg/kg up to 20 mg/kg.

In certain embodiments, after administration of the loading dose, the patient is administered one or more additional therapeutically effective doses of an HGF-Met inhibitor and an EGFR inhibitor according to a “maintenance schedule.” Exemplary maintenance schedules include, but are not limited to, administration once a week, once every two weeks, once every three weeks, once a month, once every six weeks, once every two months, once every ten weeks, once every three months, once every 14 weeks, once every four months, once every 18 weeks, once every five months, once every 22 weeks, once every six months, once every seven months, once every eight months, once every nine months, once every ten months, once every eleven months, or once every twelve months. In certain embodiments, subsequent therapeutically effective doses are administered at more frequent intervals, for example, once every two weeks to once every month. In certain such embodiments, subsequent therapeutically effective doses of an HGF-Met inhibitor and an EGFR inhibitor comprise a dose of an HGF-Met inhibitor and/or a dose of an EGFR inhibitor that fall within a lower dosing range, for example, 0.1 mg/kg up to 20 mg/kg. In certain embodiments, subsequent therapeutically effective doses are administered at less frequent intervals, for example, once every month to once every twelve months. In certain such embodiments, subsequent therapeutically effective doses of an HGF-Met inhibitor and an EGFR inhibitor comprise a dose of an HGF-Met inhibitor and/or an EGFR inhibitor that falls within a higher dosing range, for example, 20 mg/kg up to 100 mg/kg.

In certain embodiments, the route of administration of the pharmaceutical composition is in accord with known methods, e.g. orally, through-injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

In certain embodiments, intravenous administration occurs by infusion over a period of 1 to 10 hours. In certain embodiments, intravenous administration occurs by infusion over a period of 1 to 8 hours. In certain embodiments, intravenous administration occurs by infusion over a period of 2 to 7 hours. In certain embodiments, intravenous administration occurs by infusion over a period of 4 to 6 hours. In certain embodiments, intravenous administration occurs by infusion over a period of 2 to 3 hours. In certain embodiments, intravenous administration occurs by infusion over a period of 1 to 2 hours. In certain embodiments, intravenous administration occurs by infusion over a period of 0.5 to 1 hour. In certain embodiments, intravenous administration occurs by infusion over a period of 0.1 to 0.5 hours. The determination of certain appropriate infusion periods is within the skill of the art. In certain embodiments, the initial infusion is given over a period of 4 to 6 hours, with subsequent infusions delivered more quickly. In certain such embodiments, subsequent infusions are administered over a period of 1 to 6 hours.

In certain embodiments, the infusion time period for administering an antibody to EGFR in a dose of 6 mg/kg is 60 minutes±15 minutes. In certain embodiments, the infusion time period for administering an antibody to EGFR in a dose of 6 mg/kg is 90 minutes±15 for doses higher than 1000 mg. In certain embodiments, if a dose of an antibody to EGFR is well tolerated (i.e., without any serious infusion-related reactions), then subsequent IV infusions of an antibody to EGFR may be administered in a time period of 30 minutes±15 minutes. In certain embodiments, the infusion time period for administering an antibody to HGF in a dose of 10 mg/kg is 60 minutes±15 minutes. In certain embodiments, if a dose of an antibody to HGF is well tolerated (i.e., without any serious infusion-related reactions), then subsequent IV infusions of an antibody to HGF may be administered in a time period of 30 minutes 15 minutes. In certain embodiments with that dosage of antibodies, frequency of administration, and infusion time periods, for each administration, the administration of the antibody to EGFR will be administered prior to the administration of the antibody to HGF. In certain embodiments with that dosage of antibodies, frequency of administration, and infusion time periods, for each administration, the administration of the antibody to EGFR will be administered after the administration of the antibody to HGF. In certain embodiments with that dosage of antibodies, frequency of administration, and infusion time periods, for each administration, the administration of the antibody to EGFR will be administered at the same time as the administration of the antibody to HGF.

In certain embodiments, the composition may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

In certain embodiments, it may be desirable to use an HGF-Met inhibitor and an EGFR inhibitor, with or without at least one additional therapeutic agent, in an ex vivo manner. In such instances, cells, tissues and/or organs that have been removed from the patient are exposed to an HGF-Met inhibitor and an EGFR inhibitor, with or without at least one additional therapeutic agent, after which the cells, tissues and/or organs are subsequently implanted back into the patient.

In certain embodiments, an HGF-Met inhibitor and an EGFR inhibitor and/or any additional therapeutic agents can be delivered by implanting certain cells that have been genetically engineered, using methods such as those described herein, to express and secrete the polypeptides. In certain embodiments, such cells may be animal or human cells, and may be autologous, heterologous, or xenogeneic. In certain embodiments, the cells may be immortalized. In certain embodiments, in order to decrease the chance of an immunological response, the cells may be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### EXAMPLES

### Example 1

U87MG human glioblastoma tumor cells were obtained from ATCC (accession no. HTB-14). U87MG cells express HGF, Met, and EGFR. U87MG cells were expanded in culture and harvested. On Day 0, fifteen (15) 4-6 week old female nude mice (CD1 NU/NU, Charles River Laboratories) were inoculated with U-87MG cells by injecting 3×10⁶ U87MG cells in 100 microliters of phosphate-buffered saline (PBS) subcutaneously into each flank of each mouse. Xenografts (tumors) were allowed to develop for 28 days. On Day 28, the average tumor volume was 75 mm³. Each mouse had two tumors.

Each of the 15 mice was given 4 intraperitoneal injections over the next two weeks (on Days 28, 32, 35, and 39), as follows: 8 mice were injected with the HGF-Met inhibitor 2.12.1 diluted in PBS (30 μg each injection); and 7 control mice were injected with PBS. Survival of the mice and tumor volume were monitored. Tumor volume was determined using the formula (Iength×width²)/2, where length was the longest axis and width was the perpendicular axis. Measurements were made with digital calipers. Tumor volume was measured on Days 28, 32, 35, 39, 42, 46, 49, 53, 56, and 60. Where multiple groups were involved, data were analysed by analysis of variance (ANOVA) and, if appropriate, post-hoc testing with Student's t-test was undertaken.

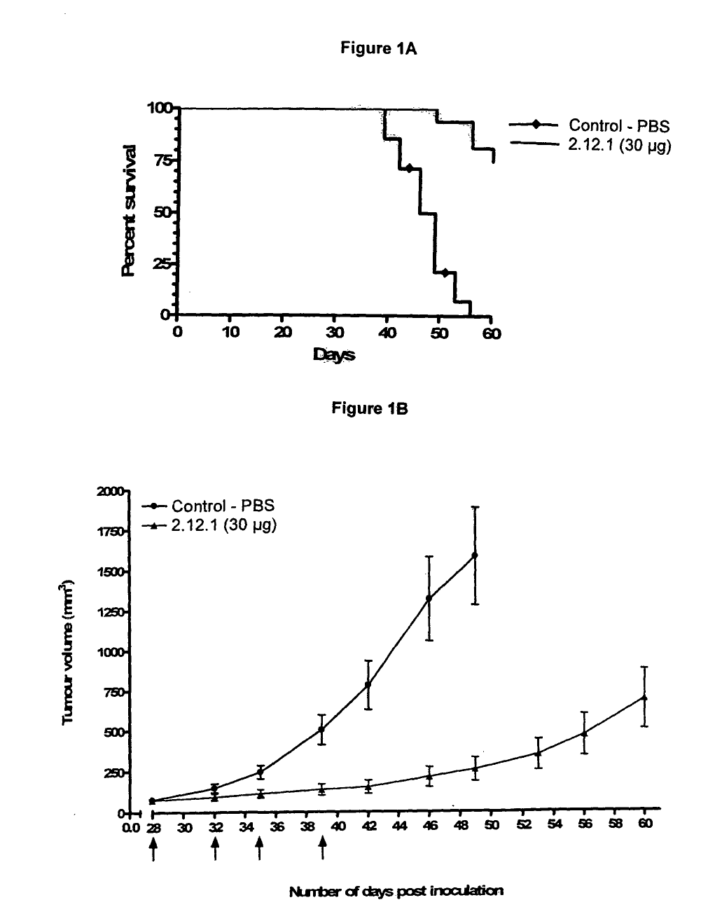

Survival data are shown in FIG. 1A, which shows a plot of the percent survival vs. days. Those data indicate that a higher percentage of the mice injected with 2.12.1 survived for 39 days or longer.

Tumor volume data are shown in FIG. 1B, which shows a plot of the average tumor volume vs. days post inoculation. Those data indicate that the average tumor volume of the mice injected with 2.12.1 was smaller than the average tumor volume of the control mice on Days 32, 35, 39, 42, 46, and 49.

### Example 2

U87MGΔ2-7 human glioblastoma tumor cells were transfected with a nucleotide sequence encoding the EGFRvIII protein (Nishikawa et al., Proc. Natl. Acad. Sci. USA 91: 7727-7731 (1994)) to generate U87MGΔ2-7 cells. U87MGΔ2-7 cells express HGF, Met, and EGFRvIII. U87MGΔ2-7 cells were expanded in culture and harvested. On Day 0, twenty-two (22) 4-6 week old female nude mice (CD1 NU/NU, Charles River Laboratories) were inoculated with U87MGΔ2-7 cells by injecting 3×10⁶ U87MGΔ2-7 cells in 100 microliters of PBS subcutaneously into each flank of each mouse. Xenografts (tumors) were allowed to develop for 7 days. On Day 7, the average tumor volume was 80 mm³. Each mouse had two tumors.

Each of the 22 mice was given 4 intraperitoneal injections over the next two weeks (on Days 7, 11, 14, and 18), as follows: 6 mice were injected with the HGF-Met inhibitor 2.12.1 diluted in PBS (30 μg each injection); 5 mice were injected with the EGFR inhibitor panitumumab diluted in PBS (1 mg each injection); 5 mice were injected with both 2.12.1 and panitumumab diluted in PBS (30 μg 2.12.1 and 1 mg panitumumab each injection); and 6 control mice were injected with PBS. Survival of the mice and tumor volume were monitored. Tumor volume was determined using the formula (Iength×width²)/2, where length was the longest axis and width was the perpendicular axis. Measurements were made with digital calipers. Tumor volume was measured on Days 7, 11, 14, 18, 22, 26, 29, 33, and 37. Where multiple groups were involved, data were analysed by analysis of variance (ANOVA) and, if appropriate, post-hoc testing with Student's t-test was undertaken.

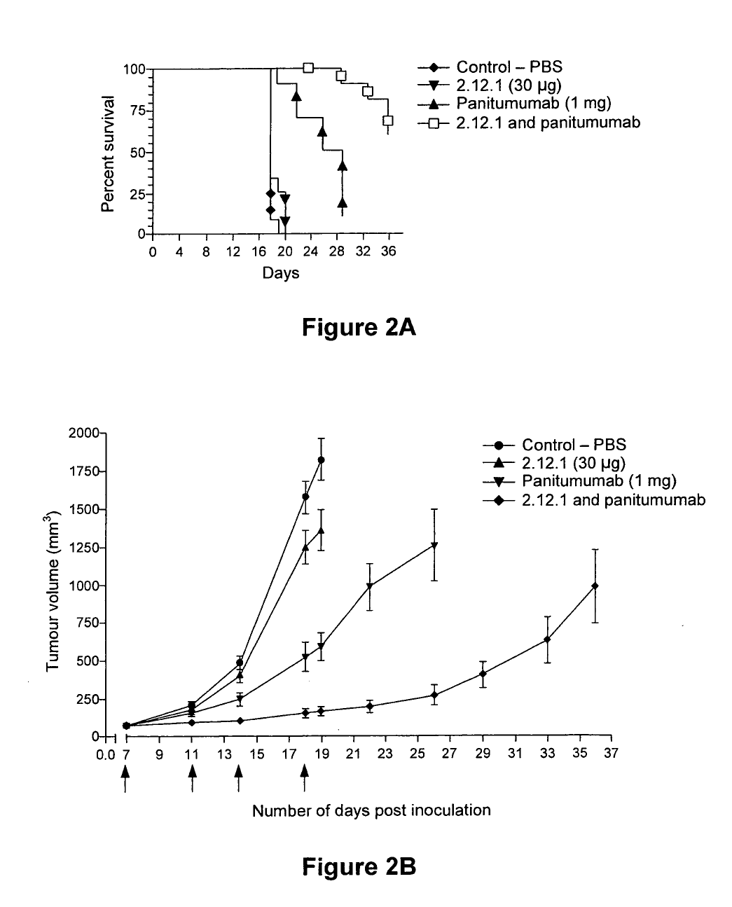

Survival data are shown in FIG. 2A. Those data indicate that a higher percentage of the mice injected with both 2.12.1 and panitumumab than the other mice survived for 19 days or longer.

Tumor volume data are shown in FIG. 2B. Those data indicate that the average tumor volume of the mice injected with both 2.12.1 and panitumumab was smaller than the average tumor volume of the other mice on Days 11, 14, 18, 19, 22, and 26.

### Example 3

U87MGΔ2-7 cells were expanded in culture and harvested. On Day 0, twenty-four (24) 4-6 week old female nude mice (CD1 NU/NU, Charles River Laboratories) were inoculated with U87MGΔ2-7 cells by injecting 3×10⁶ U87MGΔ2-7 cells in 100 microliters of PBS subcutaneously into each flank of each mouse. Xenografts (tumors) were allowed to develop for 7 days. On Day 7, the average tumor volume was 90 mm³. Each mouse had two tumors.

Each of the 24 mice was given 4 intraperitoneal injections over the next two weeks (on Days 7, 10, 14, and 17), as follows: 7 mice were injected with the HGF-Met inhibitor 2.12.1 diluted in PBS (100 μg each injection); 5 mice were injected with the EGFR inhibitor panitumumab diluted in PBS (1 mg each injection); 5 mice were injected with both 2.12.1 and panitumumab diluted in PBS (100 μg 2.12.1 and 1 mg panitumumab each injection); and 7 control mice were injected with PBS. Tumor volume was determined using the formula (Iength×width²)/2, where length was the longest axis and width was the perpendicular axis. Measurements were made with digital calipers. Tumor volume was measured on Days 7, 10, 14, 17, 21, 24, 28, 31, and 35. Where multiple groups were involved, data were analysed by analysis of variance (ANOVA) and, if appropriate, post-hoc testing with Student's t-test was undertaken.

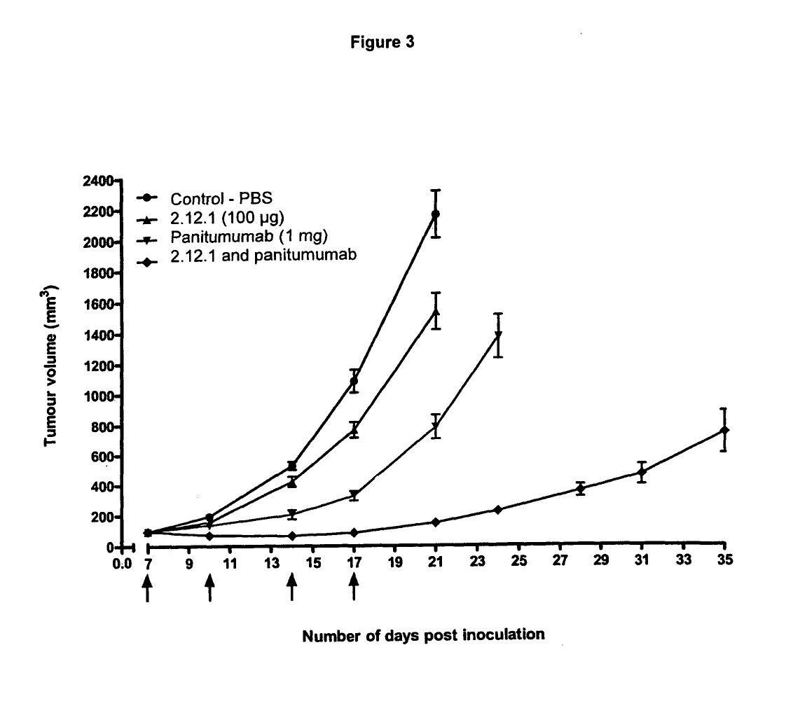

Tumor data are shown in FIG. 3. Those data indicate that the average tumor volume of the mice injected with both 2.12.1 and panitumumab was smaller than the average tumor volume of the other mice on Days 10, 14, 17, 21, and 24.

### Example 4

U87MG human glioblastoma tumor cells were transfected with a nucleotide sequence encoding the EGFR protein (Nishikawa et al., Proc. Natl. Acad. Sci. USA 91: 7727-7731 (1994)) to generate U87MG.wt cells. U87MG.wt cells express HGF and Met, and overexpress EGFR. U87MG.wt cells were expanded in culture and harvested. On Day 0, fourteen (4) 4-6 week old female nude mice (CD1 NU/NU, Charles River Laboratories) were inoculated with U87MG.wt cells by injecting 3×10⁶ U-87MG.wt cells in 100 microliters of PBS subcutaneously into each flank of each mouse. Xenografts (tumors) were allowed to develop for 12 days. On Day 12, the average tumor volume was 75 mm³. Each mouse had two tumors.

Each of the 14 mice was given 4 intraperitoneal injections over the next two weeks (on Days 12, 15, 19, and 22), as follows: 7 mice were injected with the HGF-Met inhibitor 2.12.1 diluted in PBS (30 μg each injection); and 7 control mice were injected with PBS. Tumor volume was determined using the formula (Iength×width²)/2, where length was the longest axis and width was the perpendicular axis. Measurements were made with digital calipers. Tumor volume was measured on Days 12, 15, 19, 22, 26, 29, 33, 36, 40, 43, and 47. Where multiple groups were involved, data were analysed by analysis of variance (ANOVA) and, if appropriate, post-hoc testing with Student's t-test was undertaken.

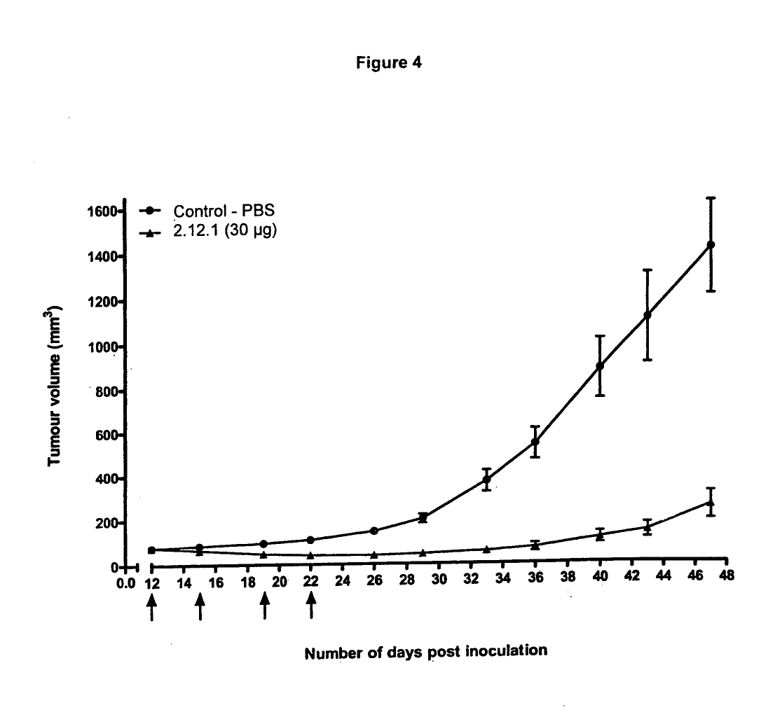

Tumor data are shown in FIG. 4. Those data indicate that the average tumor volume of the mice injected with 2.12.1 was smaller than the average tumor volume of the control mice on days on which a measurement was made except Day 12.

### Example 5

Eight white human patients (three male and five female) ranging in age from 40 to 75 years old were administered panitumumab and HGF-Met inhibitor 2.12.1. Each patient had metastatic colorectal cancer and expressed wild-type KRAS. Panitumumab and HGF-Met inhibitor 2.12.1 were administered to each patient once every two weeks. The following protocol was provided to investigators administering panitumumab and HGF-Met inhibitor 2.12.1.

Protocol

Vials containing 200 mg panitumumab in a 10 mL sterile colorless protein solution are used as the source for panitumumab administration. Doses are calculated for a 6 kg/mg dosage. The calculated volume of panitumumab from the vials is diluted in pyrogen-free 0.9% sodium chloride for injection USP/PH Eur/JP to a total volume of 100 mL. Doses higher than 1000 mg should be diluted in 150 mL sodium chloride. The final panitumumab concentration after dilution should not exceed 10 mg/mL. The diluted solution of panitumumab should not be shaken excessively and should be mixed by gentle inversion. Panitumumab is administered intraveneously (IV) by infusion pump through a peripheral line or indwelling catheter using a nonpyrogenic, low protein binding 0.2 or 0.22 micron pore size in-line filter. The infusion time period is 60 minutes±15 minutes. The infusion time period should be extended to 90 minutes±15 for doses higher than 1000 mg. If a dose of panitumumab is well tolerated (i.e., without any serious infusion-related reactions), then subsequent IV infusions of panitumumab may be administered in a time period of 30 minutes±15 minutes.

HGF-Met inhibitor 2.12.1 is provided as a frozen, sterile, clear, colorless, and preservative-free protein solution of 3.0 mL HGF-Met inhibitor 2.12.1 at a concentration of 30 mg/mL in a 10 mL vial. Doses are calculated for a 10 kg/mg dosage. The calculated volume of panitumumab from the vials is diluted in pyrogen-free 0.9% sodium chloride for injection USP/PH Eur/JP to a total volume of 100 mL. Doses higher than 1410 mg should be diluted in 150 mL sodium chloride. Doses higher than 2100 mg should be diluted in 200 mL sodium chloride. The appropriate dilutions should occur so that the final HGF-Met inhibitor 2.12.1 concentration after dilution does not exceed 14 mg/mL. The diluted solution of HGF-Met inhibitor 2.12.1 should not be shaken excessively and should be mixed by gentle inversion. Following completion of the panitumumab infusion, and proper flushing of the infusion line, the HGF-Met inhibitor 2.12.1 is administered intraveneously (IV) by infusion pump through a peripheral line or indwelling catheter. Filtration of diluted HGF-Met inhibitor 2.12.1 is not required. The infusion time period is 60 minutes±15 minutes. If a dose of HGF-Met inhibitor 2.12.1 is well tolerated (i.e., without any serious infusion-related reactions), then subsequent IV infusions of HGF-Met inhibitor 2.12.1 may be administered in a time period of 30 minutes±15 minutes.

The patients receive the dosages once every two weeks until disease progression or intolerability. Intolerability is based on the appearance of dose-limiting toxicities (DLTs).

Results

Results are provided for the first four weeks of treatment. Three patients withdrew because of PD, withdrawn consent, or death. No DLT was reported. The most common adverse events are shown in Table 1 below. More serious adverse events included acneiform dermatitis (n=1), intestinal obstruction (n=1), and cerebrovascular accident (n=1); one patient died on study.

TABLE 1
Most common Adverse Events

| | Panitumumab and |
|---|---|
| | HGF-Met inhibitor 2.12.1 |
| | (N = 6)* |
| Acneiform dermatitis | 5 (83%) |
| Worst grade of 3 | 1 (17%) |
| Worst grade of 4 | 1 (17%) |
| Pruritus | 4 (67%) |
| Constipation | 3 (50%) |
| Dry skin | 3 (50%) |
| Erythema | 3 (50%) |
| Skin fissures | 3 (50%) |
| Insomnia | 3 (50%) |

*First 6 patients completing 4 weeks of treatment

We claim:

1. A method of treating a resistant cancer in a patient comprising administering at least one HGF-Met inhibitor and at least one EGFR inhibitor.

2. The method of claim 1, wherein the cancer expresses EGFRvIII.

3. The method of claim 2, wherein at least one of the at least one HGF-Met inhibitor is a specific binding agent to HGF.

4. The method of claim 3, wherein the specific binding agent to HGF is an antibody.

5. The method of claim 4, wherein the antibody is fully human.

6. The method of claim 5, wherein the antibody is 2.12.1.

7. The method of claim 4, wherein the antibody is administered in a dose of about 2 mg/kg to about 30 mg/kg every two weeks.

8. The method of claim 2, wherein at least one of the at least one HGF-Met inhibitor is a specific binding agent to Met.

9. The method of claim 8, wherein the specific binding agent to Met is an antibody.

10. The method of claim 9, wherein the antibody is OA-5d5.

11. The method of claim 2, wherein at least one of the at least one HGF-Met inhibitor is a compound of the formula: 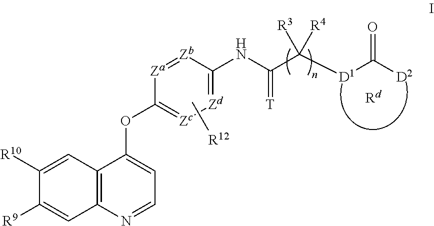
or an enantiomer, diastereomer, salt, solvate, or N-Oxide thereof
wherein T is O or S;
wherein R³ and R⁴ is each independently selected from H, C₁₋₂-alkyl, phenyl, 5-6-membered heterocyclyl, phenyl-C₁₋₂-alkyl, 5-6-membered heterocyclyl-C₁₋₂-alkyl, C₃₋₆-cycloalkyl, and C₃₋₆-cycloalkyl-C₁₋₂-alkyl; alternatively R³ and R⁴, together with the atom they are attached to, form an optionally substituted 3-6 membered ring;
wherein R⁹ and R¹⁰ is independently selected from H, cyano, hydroxy, —C(═O)NR^{a}R^{5a}, 5-6 membered heterocyclyl, —NR^{a}C(═O)—R^{5a}, R^{5a}R^{a}N—O₂S—, R^{5a}O₂SR^{a}N—, R^{5a}R^{a}N—, C₁₋₆-alkyl, amino-C₁₋₆-alkyl, C₁₋₆-alkylamino-C₁₋₆-alkyl, alkoxy-C₁₋₆-alkyl, hydroxy, aryl-C₁₋₆-alkyl, heterocyclyl-C₁₋₆-alkyl, C₁₋₆-alkoxy, halo-C₁₋₆-alkoxy, C₁₋₆-alkylamino-C₁₋₆-alkoxy, aryl-C₁₋₆-alkoxy, 5-6-membered heterocyclyl, —C₁₋₆alkoxy, C₃₋₆-cycloalkyl-C₁₋₆-alkoxy, 5-6-membered heterocyclyl(hydroxyl-C₁₋₆-alkoxy), C₃₋₆-cycloalkyl(hydroxyl-C₁₋₆-alkoxy), phenyl(hydroxyl-C₁₋₆-alkoxy), C₁₋₆-alkoxy-C₁₋₆-alkoxy, phenyloxy-C₁₋₆-alkoxy, 5-6 membered heterocyclyloxy-C₁₋₆-alkoxy, C₃₋₆-cycloalkyloxy-C₁₋₆-alkoxy, phenyloxy, 5-6-membered heterocyclyloxy, and C₃₋₆-cycloalkyloxy;
wherein each of Z^{a}, Z^{b}, Z^{c} and Z^{d} is independently selected from N or CH;
provided no more than 2 of Z^{a}, Z^{b}, Z^{c} and Z^{d} are N;
wherein n is 0, 1, 2 or 3;
wherein D¹ is selected from N or CR¹¹;
wherein D² is selected from NR¹³, O, or CHR¹¹; provided either D¹ is N or D² is NR¹³;
wherein ring R^{d} including 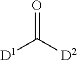
forms an optionally substituted optionally benzo-fused 4-7 membered heterocyclic moiety,
wherein R¹¹ is selected from H, halo, C₁₋₄-alkyl, C₁₋₄-haloalkyl, C₁₋₄-hydroxyalkyl, —NH₂, —OR¹², alkoxycarbonyl, —CO₂H, —CONR³R^{5a}, (C₁-C₃)alkylamino, di(C₁-C₆)alkylamino, (C₁-C₃)hydroxyalkylamino, (C₁-C₃)alkylamino-(C₁-C₃)alkylamino, C₁₋₃-alkoxy-C₁₋₃-alkyl, C₁₋₃-alkylamino-C₁₋₃-alkyl, C₁₋₃-alkylthio-C₁₋₃-alkyl, optionally substituted phenyl-C₁₋₃-alkyl, 5-6 membered heterocyclyl-C₁₋₃-alkyl, C₃₋₆-cycloalkyl-C₁₋₃-alkyl, optionally substituted phenyl, optionally substituted 5-6 membered heterocyclyl, and C₃₋₆-cycloalkyl;
wherein R^{a} is selected from H, alkyl, heterocyclyl, aryl, arylalkyl, heterocyclylalkyl, cycloalkyl, cycloalkylalkyl, alkenyl and alkynyl;
wherein R^{5a} is selected from H, alkyl, haloalkyl, arylalkyl, heterocyclylalkyl, cycloalkylalkyl, aryl, heterocyclyl, alkenyl, alkynyl and cycloalkyl;
wherein R¹² is selected from H, halo, C₁₋₂-alkyl and methoxy;
wherein R¹³ is selected from H, alkyl, haloalkyl, optionally substituted phenylalkyl, optionally substituted 5-10 membered heterocyclylalkyl, cycloalkylalkyl, optionally substituted phenyl or naphthyl, optionally substituted 5-10 membered heterocyclyl and cycloalkyl.

12. The method of claim 2, wherein at least one of the at least one HGF-Met inhibitor is selected from:
N-(4-(6,7-dimethoxyquinolin-4-yloxy)-3-fluorophenyl)-1-methyl-3-oxo-2-phenyl-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(4-(6,7-dimethoxyquinolin-4-yloxy)-3-fluorophenyl)-5-((ethyl(methyl)amino)methyl)-1-methyl-3-oxo-2-phenyl-2,3-dihydro-1H-pyrazole-4-carboxamide;
N-(4-(6,7-dimethoxyquinolin-4-yloxy)-3-fluorophenyl)-5-((dimethylamino)methyl)-1-methyl-3-oxo-2-phenyl-2,3-dihydro-1H-pyrazole-4-carboxamide;
5-(aminomethyl)-N-(3-fluoro-4-(7-methoxyquinolin-4-yloxy)phenyl)-1-methyl-3-oxo-2-phenyl-2,3-dihydro-1H-pyrazole-4-carboxamide;
tert-butyl (4-((3-fluoro-4-(7-methoxyquinolin-4-yloxy)phenyl)carbamoyl)-1-methyl-3-oxo-2-phenyl-2,3-dihydro-1H-pyrazol-5-yl)methylcarbamate,
N-(5-(7-methoxyquinolin-4-yloxy)pyridin-2-yl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyrrolidin-1-ylmethyl)-2,3-dihydro-1H-pyrazole-4-carboxamide;
N-(3-fluoro-4-(7-methoxyquinolin-4-yloxy)phenyl)-5-methyl-3-oxo-2-phenyl-1-((tetrahydrofuran-2-yl)methyl)-2,3-dihydro-1H-pyrazole-4-carboxamide;
5-((ethyl(methyl)amino)methyl)-N-(3-fluoro-4-(7-methoxyquinolin-4-yloxy)phenyl)-1-methyl-3-oxo-2-phenyl-2,3-dihydro-1H-pyrazole-4-carboxamide;
2-benzyl-N-(5-(7-methoxyquinolin-4-yloxy)pyridin-2-yl)-1-methyl-3-oxo-5-(pyridin-4-yl)-2,3-dihydro-1H-pyrazole-4-carboxamide;
2-benzyl-N-(3-fluoro-4-(7-methoxyquinolin-4-yloxy)phenyl)-1-methyl-3-oxo-5-(pyridin-4-yl)-2,3-dihydro-1H-pyrazole-4-carboxamide;
(S)—N-(5-(7-methoxyquinolin-4-yloxy)pyridin-2-yl)-1-methyl-3-oxo-2-(1-phenylethyl)-5-(pyridin-4-yl)-2,3-dihydro-1H-pyrazole-4-carboxamide;
(S)—N-(3-fluoro-4-(7-methoxyquinolin-4-yloxy)phenyl)-1-methyl-3-oxo-2-(1-phenylethyl)-5-(pyridin-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-4-yl)-2,3-dihydro-1H-pyrazole-4-carboxamide;
N-(5-(6,7-dimethoxyquinolin-4-yloxy)pyridin-2-yl)-1-methyl-3-oxo-2-phenyl-5-(pyridin-4-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(pyridin-2-yl)-2,3-dihydro-1H-pyrazole-4-carboxamide;
N-(5-(7-methoxyquinolin-4-yloxy)pyridin-2-yl)-1-methyl-3-oxo-2-phenyl-5-(pyridin-2-yl)-2,3-dihydro-1H-pyrazole-4-carboxamide;
N-(3-fluoro-4-(7-methoxyquinolin-4-yloxy)phenyl)-1-methyl-3-oxo-2-phenyl-5-(tetrahydro-2H-pyran-4-yl)-2,3-dihydro-1H-pyrazole-4-carboxamide;
N-(5-(7-methoxyquinolin-4-yloxy)pyridin-2-yl)-1-methyl-3-oxo-2-phenyl-5-(tetrahydro-2H-pyran-4-yl)-2,3-dihydro-1H-pyrazole-4-carboxamide;
1-Methyl-N-(5-((7-(methyloxy)-4-quinolinyl)oxy)-2-pyridinyl)-5-(2-methyl-1,3-thiazol-4-yl)-3-oxo-2-phenyl-2,3-dihydro-1H-pyrazole-4-carboxamide;
N-(5-((6,7-bis(methyloxy)-4-quinolinyl)oxy)-2-pyridinyl)-1-methyl-5-(5-methyl-3-isoxazolyl)-3-oxo-2-phenyl-2,3-dihydro-1H-pyrazole-4-carboxamide;
1-methyl-5-(5-methyl-3-isoxazolyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(5-methyl-3-isoxazolyl)-3-oxo-2-phenyl-2,3-dihydro-1H-pyrazole-4-carboxamide;
1-methyl-N-(5-((7-(methyloxy)-4-quinolinyl)oxy)-2-pyridinyl)-3-oxo-2-phenyl-5-(2-pyrazinyl)-2,3-dihydro-1H-pyrazole-4-carboxamide;
N-(3-fluoro-4-((7-(methyloxy)-4-quinolinyl)oxy)phenyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-3-oxo-2-phenyl-5-(2-pyrazinyl)-2,3-dihydro-1H-pyrazole-4-carboxamide;
N-(5-((6,7-bis(methyloxy)-4-quinolinyl)oxy)-2-pyridinyl)-1-methyl-5-(2-methyl-1,3-thiazol-4-yl)-3-oxo-2-phenyl-2,3-dihydro-1H-pyrazole-4-carboxamide;
N-(3-fluoro-4-((7-(methyloxy)-4-quinolinyl)oxy)phenyl)-1-methyl-5-(2-methyl-1,3-thiazol-4-yl)-3-oxo-2-phenyl-2,3-dihydro-1H-pyrazole-4-carboxamide;
N-(4-(6,7-dimethoxyquinolin-4-yloxy)-3-fluorophenyl)-N,1,5-trimethyl-3-oxo-2-phenyl-2,3-dihydro-1H-pyrazole-4-carboxamide;
2-(3-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(3-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
N-(4-(6,7-dimethoxyquinolin-4-yloxy)-3-fluorophenyl)-1,5-dimethyl-3-oxo-2-p-tolyl-2,3-dihydro-1H-pyrazole-4-carboxamide;
N-(4-(6,7-dimethoxyquinolin-4-yloxy)-3-fluorophenyl)-2-(4-fluorophenyl)-1,5-dimethyl-3-oxo-2,3-dihydro-1H-pyrazole-4-carboxamide;
N-(5-(6,7-dimethoxyquinolin-4-yloxy)pyridine-2-yl)-1,5-dimethyl-3-oxo-2-p-tolyl-2,3-dihydro-1H-pyrazole-4-carboxamide;
N-(5-(6,7-dimethoxyquinolin-4-yloxy)pyridin-2-yl)-2-(4-fluorophenyl)-1,5-dimethyl-3-oxo-2,3-dihydro-1H-pyrazole-4-carboxamide;
2-(3-chlorophenyl)-N-(5-(7-methoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1,5-dimethyl-3-oxo-2-p-tolyl-2,3-dihydro-1H-pyrazole-4-carboxamide;
2-(2-chlorophenyl)-N-(5-(6,7-dimethoxyquinolin-4-yloxy)pyridin-2-yl)-1,5-dimethyl-3-oxo-2,3-dihydro-1H-pyrazole-4-carboxamide;
2-(2-chlorophenyl)-N-(4-(6,7-dimethoxyquinolin-4-yloxy)-3-fluorophenyl)-1,5-dimethyl-3-oxo-2,3-dihydro-1H-pyrazole-4-carboxamide;
2-(2-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(3-fluoro-4-(7-methoxyquinolin-4-yloxy)phenyl)-2-(4-fluorophenyl)-1,5-dimethyl-3-oxo-2,3-dihydro-1H-pyrazole-4-carboxamide;
2-(3-chlorophenyl)-N-(3-fluoro-4-(7-methoxyquinolin-4-yloxy)phenyl)-1,5-dimethyl-3-oxo-2,3-dihydro-1H-pyrazole-4-carboxamide;
N-(6-(6,7-dimethoxyquinolin-4-yloxy)pyridin-3-yl)-1,5-dimethyl-3-oxo-2-phenyl-2,3-dihydro-1H-pyrazole-4-carboxamide;
N-(2-chloro-4-(6,7-dimethoxyquinolin-4-yloxy)phenyl)-1,5-dimethyl-3-oxo-2-phenyl-2,3-dihydro-1H-pyrazole-4-carboxamide;
2-benzyl-N-(4-(6,7-dimethoxyquinolin-4-yloxy)-3-fluorophenyl)-1,5-dimethyl-3-oxo-2,3-dihydro-1H-pyrazole-4-carboxamide;
2-benzyl-N-(5-(6,7-dimethoxyquinolin-4-yloxy)pyridin-2-yl)-1,5-dimethyl-3-oxo-2,3-dihydro-1H-pyrazole-4-carboxamide;
N-(5-(7-methoxyquinolin-4-yloxy)pyridin-2-yl)-1-methyl-3-oxo-2-phenyl-2,3-dihydro-1H-pyrazole-4-carboxamide;
N-(4-(6,7-dimethoxyquinolin-4-yloxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-3-oxo-1-(2-oxobutyl)-2-phenyl-2,3-dihydro-1H-pyrazole-4-carboxamide;
N-(3-fluoro-4-(7-methoxyquinolin-4-yloxy)phenyl)-5-methyl-1-(3-methyl-2-oxobutyl)-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-((2R,3R)-3-hydroxybutan-2-yl)-5-methyl-3-oxo-2-phenyl-2,3-dihydro-1H-pyrazole-4-carboxamide;
1-((2R,3R)-3-hydroxybutan-2-yl)-N-(5-(7-methoxyquinolin-4-yloxy)pyridin-2-yl)-5-methyl-3-oxo-2-phenyl-2,3-dihydro-1H-pyrazole-4-carboxamide;
(S)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(R)-1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
(S)—N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-((3-methyl-2-oxooxazolidin-5-yl)methyl)-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-(methylamino)propyl)-5-methyl-3-oxo-2-phenyl-2,3-dihydro-1H-pyrazole-4-carboxamide;
1-(3-chloro-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylbutyl)-5-methyl-3-oxo-2-phenyl-2,3-dihydro-1H-pyrazole-4-carboxamide;
1-(2-hydroxy-3-methylbutyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-methylbutyl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-3-morpholinopropyl)-5-methyl-3-oxo-2-phenyl-2,3-dihydro-1H-pyrazole-4-carboxamide;
N-(3-fluoro-4-(7-methoxyquinolin-4-yloxy)phenyl)-5-methyl-1-(oxazolidin-5-ylmethyl)-3-oxo-2-phenyl-2,3-dihydro-1H-pyrazole-4-carboxamide;
(S)—N-(3-fluoro-4-(7-methoxyquinolin-4-yloxy)phenyl)-1-(2-hydroxybutyl)-5-methyl-3-oxo-2-phenyl-2,3-dihydro-1H-pyrazole-4-carboxamide;
1-(3-amino-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
1-(2-hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide;
N-(3-fluoro-4-(7-methoxyquinolin-4-yloxy)phenyl)-1-(2-hydroxy-2-methylpropyl)-5-methyl-3-oxo-2-phenyl-2,3-dihydro-1H-pyrazole-4-carboxamide;
(R)-1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
1-(3-(dimethylamino)-2-hydroxypropyl)-N-(3-fluoro-4-(7-methoxyquinolin-4-yloxy)phenyl)-5-methyl-3-oxo-2-phenyl-2,3-dihydro-1H-pyrazole-4-carboxamide;
(R)—N-(3-fluoro-4-(7-methoxyquinolin-4-yloxy)phenyl)-1-(2-hydroxypropyl)-5-methyl-3-oxo-2-phenyl-2,3-dihydro-1H-pyrazole-4-carboxamide;
(R)—N-(4-(6,7-dimethoxyquinolin-4-yloxy)-3-fluorophenyl)-1-(2-hydroxypropyl)-5-methyl-3-oxo-2-phenyl-2,3-dihydro-1H-pyrazole-4-carboxamide;
1-(2-hydroxypropyl)-N-(5-(7-methoxyquinolin-4-yloxy)pyridin-2-yl)-5-methyl-3-oxo-2-phenyl-2,3-dihydro-1H-pyrazole-4-carboxamide;
N-(5-(6,7-dimethoxyquinolin-4-yloxy)pyridin-2-yl)-1-(2-hydroxy-2-methylpropyl)-5-methyl-3-oxo-2-phenyl-2,3-dihydro-1H-pyrazole-4-carboxamide;
(R)-2-(3-chlorophenyl)-N-(3-fluoro-4-(7-methoxyquinolin-4-yloxy)phenyl)-1-(2-hydroxypropyl)-5-methyl-3-oxo-2,3-dihydro-1H-pyrazole-4-carboxamide;
(R)-2-(3-chlorophenyl)-1-(2-hydroxypropyl)-N-(5-(7-methoxyquinolin-4-yloxy)pyridin-2-yl)-5-methyl-3-oxo-2,3-dihydro-1H-pyrazole-4-carboxamide;
(R)—N-(3-fluoro-4-(7-methoxyquinolin-4-yloxy)phenyl)-2-(4-fluorophenyl)-1-(2-hydroxypropyl)-5-methyl-3-oxo-2,3-dihydro-1H-pyrazole-4-carboxamide
1-(2-hydroxy-2-methylpropyl)-N-(5-(1-oxo-7-methoxyquinolin-4-yloxy)pyridin-2-yl)-5-methyl-3-oxo-2-phenyl-2,3-dihydro-1H-pyrazole-4-carboxamide;
N-(3-Fluoro-4-(7-hydroxyquinolin-4-yloxy)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-N-(5-(7-hydroxyquinolin-4-yloxy)pyridin-2-yl)-5-methyl-3-oxo-2-phenyl-2,3-dihydro-1H-pyrazole-4-carboxamide;
N-(4-(6-Ethyl-7-methoxyquinolin-4-yloxy)-3-fluorophenyl)-1,5-dimethyl-3-oxo-2-phenyl-2,3-dihydro-1H-pyrazole-4-carboxamide;
N-(3-Fluoro-4-(7-methoxyquinolin-4-yloxy)phenyl)-1,5-dimethyl-3-oxo-2-phenyl-2,3-dihydro-1H-pyrazole-4-carboxamide;
N-(3-fluoro-4-(7-Methoxyquinolin-4-yloxy)phenyl)-1,2-dimethyl-3-oxo-5-phenyl-2,3-dihydro-1H-pyrazole-4-carboxamide;
N-(5-(7-Methoxyquinolin-4-yloxy)pyridin-2-yl)-1,2-dimethyl-3-oxo-5-phenyl-2,3-dihydro-1H-pyrazole-4-carboxamide;
N-(4-(6,7-Dimethoxyquinolin-4-yloxy)-3-fluorophenyl)-1,2-dimethyl-3-oxo-5-phenyl-2,3-dihydro-1H-pyrazole-4-carboxamide;
N-(5-(7-Methoxyquinolin-4-yloxy)pyridin-2-yl)-1,5-dimethyl-3-oxo-2-phenyl-2,3-dihydro-1H-pyrazole-4-carboxamide;
(R)-1-(2-Hydroxypropyl)-N-(5-(7-methoxyquinolin-4-yloxy)pyridin-2-yl)-2-methyl-3-oxo-5-phenyl-2,3-dihydro-1H-pyrazole-4-carboxamide;
(R)—N-(3-Fluoro-4-(7-methoxyquinolin-4-yloxy)phenyl)-1-(2-hydroxypropyl)-2-methyl-3-oxo-5-phenyl-2,3-dihydro-1H-pyrazole-4-carboxamide
(S)—N-(3-fluoro-4-(6-methoxyquinolin-4-yloxy)phenyl)-1-(2-hydroxypropyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide
1-(2-(1,3-dioxo-1,3-dihydro-2H-isoindol-2-yl)ethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-(2-aminoethyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-(phenylmethyl)-2,3-dihydro-1H-pyrazole-4-carboxamide
1-benzyl-N-(3-fluoro-4-(7-methoxyquinolin-4-yloxy)phenyl)-5-methyl-3-oxo-2-phenyl-2,3-dihydro-1H-pyrazole-4-carboxamide;
5-methyl-1-(2-(methyloxy)ethyl)-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-(methyloxy)ethyl)-3-oxo-2-phenyl-2,3-dihydro-1H-pyrazole-4-carboxamide;
1-(2-hydroxyethyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
1-((2R)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
(S)-1-(2-(dimethylamino)propyl)-N-(3-fluoro-4-(7-methoxyquinolin-4-yloxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-(2-(1-pyrrolidinyl)ethyl)-2,3-dihydro-1H-pyrazole-4-carboxamide;
1-((2S)-2-fluoropropyl)-5-methyl-N-(5-((7-(methyloxy)-4-quinolinyl)oxy)-2-pyridinyl)-3-oxo-2-phenyl-2,3-dihydro-1H-pyrazole-4-carboxamide;
N-(3-fluoro-4-((7-(methyloxy)-4-quinolinyl)oxy)phenyl)-1-((2S)-2-fluoropropyl)-5-methyl-3-oxo-2-phenyl-2,3-dihydro-1H-pyrazole-4-carboxamide;
1-((2S)-2-(acetylamino)propyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-aminopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
1-((2S)-2-azidopropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxyethyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-1-((2R)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
5-methyl-N-(5-((7-(methyloxy)-4-quinolinyl)oxy)-2-pyridinyl)-1-(2-methylpropyl)-3-oxo-2-phenyl-2,3-dihydro-1H-pyrazole-4-carboxamide;
5-methyl-N-(5-((7-(methyloxy)-4-quinolinyl)oxy)-2-pyridinyl)-3-oxo-2-phenyl-1-propyl-2,3-dihydro-1H-pyrazole-4-carboxamide;
N-(3-fluoro-4-((7-(methyloxy)-4-quinolinyl)oxy)phenyl)-5-methyl-3-oxo-1-(2-oxopropyl)-2-phenyl-2,3-dihydro-1H-pyrazole-4-carboxamide;
1-(2,3-dihydroxy-2-methylpropyl)-N-(3-fluoro-4-((7-(methyloxy)-4-quinolinyl)oxy)phenyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-1-(2-hydroxypropyl)-5-methyl-3-oxo-2-phenyl-2,3-dihydro-1H-pyrazole-4-carboxamide;
N-(4-((6,7-bis(methyloxy)-4-quinazolinyl)oxy)-3-fluorophenyl)-1-(2-hydroxy-2-methylpropyl)-5-methyl-3-oxo-2-phenyl-2,3-dihydro-1H-pyrazole-4-carboxamide;
N-(3-fluoro-4-((7-(methyloxy)-4-quinolinyl)oxy)phenyl)-5-methyl-1-(2-methyl-2-propen-1-yl)-3-oxo-2-phenyl-2,3-dihydro-1H-pyrazole-4-carboxamide;
N-(4-((6,7-bis(methyloxy)-4-quinolinyl)oxy)-3-fluorophenyl)-1-((2S)-2-hydroxypropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-3-oxo-1-(2-oxopropyl)-2-phenyl-2,3-dihydro-1H-pyrazole-4-carboxamide;
N-(4-((6,7-bis(methyloxy)-4-quinolinyl)oxy)-3-fluorophenyl)-1-(2,3-dihydroxy-2-methylpropyl)-5-methyl-3-oxo-2-phenyl-2,3-dihydro-1H-pyrazole-4-carboxamide;
N-(4-((6,7-bis(methyloxy)-4-quinolinyl)oxy)-3-fluorophenyl)-5-methyl-1-(2-methyl-2-propen-1-yl)-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-propyl-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-2,3-dihydro-1H-pyrazole-4-carboxamide;
N-(5-((6,7-bis(methyloxy)-4-quinolinyl)oxy)-2-pyridinyl)-5-methyl-3-oxo-2-phenyl-1-(2-propen-1-yl)-2,3-dihydro-1H-pyrazole-4-carboxamide;
N-(4-((6,7-bis(methyloxy)-1-oxido-4-quinolinyl)oxy)-3-fluorophenyl)-5-methyl-3-oxo-2-phenyl-1-(2-propen-1-yl)-2,3-dihydro-1H-pyrazole-4-carboxamide;
N-(4-((6,7-bis(methyloxy)-4-quinolinyl)oxy)-3-fluorophenyl)-5-methyl-3-oxo-2-phenyl-1-(phenylmethyl)-2,3-dihydro-1H-pyrazole-4-carboxamide;
4-(6,7-Dimethoxyquinolin-4-yloxy)-3-fluoro-N-(5-oxo-1-phenyl-2,5-dihydro-1H-pyrazol-3-yl)benzamide;
4-(6,7-Dimethoxyquinolin-4-yloxy)-N-((1,2-dimethyl-5-oxo-3-phenyl-2,5-dihydro-1H-pyrazol-4-yl)methyl)-3-fluorobenzamide;
4-(6,7-Dimethoxyquinolin-4-yloxy)-N-(2,3-dimethyl-5-oxo-1-phenyl-2,5-dihydro-1H-pyrazol-4-yl)-3-fluorobenzamide
4-(6,7-Dimethoxyquinolin-4-yloxy)-N-((2,3-dimethyl-5-oxo-1-phenyl-2,5-dihydro-1H-pyrazol-4-yl)methyl)-3-fluorobenzamide;
1-Benzyl-N-(5-(7-methoxyquinolin-4-yloxy)pyridin-2-yl)-2-oxo-1,2-dihydropyrazolo[1,5-a]pyridine-3-carboxamide;
4-((5-(6,7-Dimethoxyquinolin-4-yloxy)pyridin-2-ylamino)methyl)-1,5-dimethyl-2-phenyl-1,2-dihydropyrazol-3-one;
N-(3-fluoro-4-(2-(3-methyl-1,2,4-oxadiazol-5-yl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-((2-(1-methyl-1H-imidazol-5-yl)thieno[3,2-b]pyridin-7-yl)oxy)phenyl)-1-(2-hydroxy-2-methylpropyl)-5-methyl-3-oxo-2-phenyl-2,3-dihydro-1H-pyrazole-4-carboxamide,
N-(3-fluoro-4-((2-(1-methyl-1H-imidazol-5-yl)thieno[3,2-b]pyridin-7-yl)oxy)phenyl)-1-((2R)-2-hydroxypropyl)-5-methyl-3-oxo-2-phenyl-2,3-dihydro-1H-pyrazole-4-carboxamide;
N-(3-fluoro-4-(7H-pyrrolo[2,3-d]pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(1H-pyrrolo[2,3-b]pyridin-4-yloxy)phenyl)-1-(2-hydroxy-2-methylpropyl)-5-methyl-3-oxo-2-phenyl-2,3-dihydro-1H-pyrazole-4-carboxamide;
Methyl(6-((4-(((1-(2-hydroxy-2-methylpropyl)-5-methyl-3-oxo-2-phenyl-2,3-dihydro-1H-pyrazol-4-yl)carbonyl)amino)phenyl)oxy)-1H-benzimidazol-2-yl)carbamate;
N-(4-(2-(azetidine-1-carbonyl)thieno[3,2-b]pyridin-7-yloxy)-3-fluorophenyl)-5-methyl-3-oxo-2-phenyl-1-propyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
N-(3-fluoro-4-(2-(1-methylpiperazine-4-carbonyl)thieno[3,2-b]pyridin-7-yloxy)phenyl)-1-(2-hydroxy-2-methylpropyl)-5-methyl-3-oxo-2-phenyl-2,3-dihydro-1H-pyrazole-4-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(4-(2-(3-(dimethylamino)pyrrolidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N,N-dimethylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide;
N-(2-(dimethylamino)ethyl)-7-(2-fluoro-4-(1-(2-hydroxy-2-methylpropyl)-5-methyl-3-oxo-2-phenyl-2,3-dihydro-1H-pyrazole-4-carboxamido)phenoxy)-N-methylthieno[3,2-b]pyridine-2-carboxamide;
7-(2-fluoro-4-(1-(2-hydroxy-2-methylpropyl)-5-methyl-3-oxo-2-phenyl-2,3-dihydro-1H-pyrazole-4-carboxamido)phenoxy)-N-(2-methoxyethyl)thieno[3,2-b]pyridine-2-carboxamide;
N-(4-(2-(azetidine-1-carbonyl)thieno[3,2-b]pyridin-7-yloxy)-3-fluorophenyl)-1-(2-hydroxy-2-methylpropyl)-5-methyl-3-oxo-2-phenyl-2,3-dihydro-1H-pyrazole-4-carboxamide;
N-cyclopropyl-7-(2-fluoro-4-(1-(2-hydroxy-2-methylpropyl)-5-methyl-3-oxo-2-phenyl-2,3-dihydro-1H-pyrazole-4-carboxamido)phenoxy)thieno[3,2-b]pyridine-2-carboxamide
7-(2-fluoro-4-(5-methyl-3-oxo-2-phenyl-1-propyl-2,3-dihydro-1H-pyrazole-4-carboxamido)phenoxy)thieno[3,2-b]pyridine-2-carboxamide;
N-(3-fluoro-4-(6-(pyrrolidine-1-carboxamido)pyrimidin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(6-(pyrrolidine-1-carboxamido)pyrimidin-4-yloxy)phenyl)-5-methyl-3-oxo-2-phenyl-1-propyl-2,3-dihydro-1H-pyrazole-4-carboxamide;
N-(6-(4-(1,5-dimethyl-3-oxo-2-phenyl-2,3-dihydro-1H-pyrazole-4-carboxamido)-2-fluorophenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)morpholine-4-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)piperidine-1-carboxamide;
N-(6-(2-fluoro-4-(5-methyl-3-oxo-2-phenyl-1-propyl-2,3-dihydro-1H-pyrazole-4-carboxamido)phenoxy)pyrimidin-4-yl)-4-methylpiperazine-1-carboxamide;
(R)—N-(4-(6-(3-(dimethylamino)pyrrolidine-1-carboxamido)pyrimidin-4-yloxy)-3-fluorophenyl)-5-methyl-3-oxo-2-phenyl-1-propyl-2,3-dihydro-1H-pyrazole-4-carboxamide;
(R)—N-(4-(6-aminopyrimidin-4-yloxy)-3-fluorophenyl)-1-(2-hydroxypropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1,5-dimethyl-3-oxo-2-phenyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)piperidine-1-carboxamide;
(R)—N-(4-(2-(3-(dimethylamino)pyrrolidine-1-carboxamido)pyridin-4-yloxy)-3-fluorophenyl)-1,5-dimethyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-1-(2-hydroxy-2-methylpropyl)-5-methyl-3-oxo-2-phenyl-2,3-dihydro-1H-pyrazole-4-carboxamide;
N-(3-fluoro-4-(2-(pyrrolidine-1-carboxamido)pyridin-4-yloxy)phenyl)-5-methyl-3-oxo-2-phenyl-1-propyl-2,3-dihydro-1H-pyrazole-4-carboxamide;
N-(4-(4-(1,5-dimethyl-3-oxo-2-phenyl-2,3-dihydro-1H-pyrazole-4-carboxamido)-2-fluorophenoxy)pyridin-2-yl)morpholine-4-carboxamide;
N-(4-(2-fluoro-4-(1-(2-hydroxy-2-methylpropyl)-5-methyl-3-oxo-2-phenyl-2,3-dihydro-1H-pyrazole-4-carboxamido)phenoxy)pyridin-2-yl)piperidine-1-carboxamide;
5-methyl-N-(4-((7-(methyloxy)-4-quinolinyl)methyl)phenyl)-3-oxo-2-phenyl-1-propyl-2,3-dihydro-1H-pyrazole-4-carboxamide;
N-(4-(hydroxy(7-methoxyquinolin-4-yl)methyl)phenyl)-5-methyl-3-oxo-2-phenyl-1-propyl-2,3-dihydro-1H-pyrazole-4-carboxamide, 1,5-dimethyl-N-(5-((7-(methyloxy)-4-quinolinyl)oxy)-2-pyrimidinyl)-3-oxo-2-phenyl-2,3-dihydro-1H-pyrazole-4-carboxamide;
5-methyl-N-(4-((7-(methyloxy)-4-quinolinyl)sulfinyl)phenyl)-3-oxo-2-phenyl-1-propyl-2,3-dihydro-1H-pyrazole-4-carboxamide
1-(2-hydroxy-2-methylpropyl)-5-methyl-N-(4-((7-(methyloxy)-4-quinolinyl)thio)phenyl)-3-oxo-2-phenyl-2,3-dihydro-1H-pyrazole-4-carboxamide;
5-methyl-N-(4-((7-(methyloxy)-4-quinolinyl)thio)phenyl)-3-oxo-2-phenyl-1-propyl-2,3-dihydro-1H-pyrazole-4-carboxamide;
5-methyl-N-(3-((7-(methyloxy)-4-quinolinyl)oxy)propyl)-3-oxo-2-phenyl-1-propyl-2,3-dihydro-1H-pyrazole-4-carboxamide;
5-methyl-N-(trans-4-((7-(methyloxy)-4-quinolinyl)oxy)cyclohexyl)-3-oxo-2-phenyl-1-propyl-2,3-dihydro-1H-pyrazole-4-carboxamide;
5-methyl-N-(cis-4-((7-(methyloxy)-4-quinolinyl)oxy)cyclohexyl)-3-oxo-2-phenyl-1-propyl-2,3-dihydro-1H-pyrazole-4-carboxamide;
1-(2-hydroxy-2-methylpropyl)-5-methyl-N-(trans-4-((7-(methyloxy)-4-quinolinyl)oxy)cyclohexyl)-3-oxo-2-phenyl-2,3-dihydro-1H-pyrazole-4-carboxamide;
5-methyl-N-(4-((7-(methyloxy)-4-quinolinyl)amino)phenyl)-3-oxo-2-phenyl-1-propyl-2,3-dihydro-1H-pyrazole-4-carboxamide;
5-methyl-N-(5-((7-(methyloxy)-4-quinolinyl)oxy)-2-pyrimidinyl)-3-oxo-2-phenyl-1-propyl-2,3-dihydro-1H-pyrazole-4-carboxamide;
N-(3-fluoro-4-((7-(methyloxy)-4-quinolinyl)amino)phenyl)-1-(2-hydroxy-2-methylpropyl)-5-methyl-3-oxo-2-phenyl-2,3-dihydro-1H-pyrazole-4-carboxamide;
1-(2-hydroxy-2-methylpropyl)-5-methyl-4-((7-((7-(methyloxy)-4-quinolinyl)oxy)-2,3-dihydro-4H-1,4-benzoxazin-4-yl)carbonyl)-2-phenyl-1,2-dihydro-3H-pyrazol-3-one;
1-(2-hydroxy-2-methylpropyl)-5-methyl-N-(4-((7-(methyloxy)-4-quinolinyl)amino)phenyl)-3-oxo-2-phenyl-2,3-dihydro-1H-pyrazole-4-carboxamide;
N-(4-(6,7-dimethoxyquinolin-4-yloxy)-3-fluorophenyl)-3-hydroxy-2-(1-oxoisoindolin-2-yl)propanamide;
N-(4-(6,7-dimethoxyquinolin-4-yloxy)-3-fluorophenyl)-2-(1-oxoisoindolin-2-yl)acetamide;
N-(4-(6,7-dimethoxyquinolin-4-yloxy)-3-fluorophenyl)-2-oxo-1,5-diphenyl-1,2-dihydropyridine-3-carboxamide;
N-(5-((6,7-bis(methyloxy)-4-quinolinyl)oxy)-2-pyridinyl)-6-oxo-1-(phenylmethyl)-1,1′,2′,3′,6,6′-hexahydro-3,4′-bipyridine-5-carboxamide;
N-(5-((6,7-bis(methyloxy)-4-quinolinyl)oxy)-2-pyridinyl)-6-oxo-1-(phenylmethyl)-1,6-dihydro-3,3′-bipyridine-5-carboxamide;
N-(5-((6,7-bis(methyloxy)-4-quinolinyl)oxy)-2-pyridinyl)-6′-oxo-1′-(phenylmethyl)-1′,6′-dihydro-2,3′-bipyridine-5′-carboxamide;
N-(5-((6,7-bis(methyloxy)-4-quinolinyl)oxy)-2-pyridinyl)-2-oxo-1-(phenylmethyl)-5-(2-thienyl)-1,2-dihydro-3-pyridinecarboxamide;
N-(5-((6,7-bis(methyloxy)-4-quinolinyl)oxy)-2-pyridinyl)-2-oxo-1-(phenylmethyl)-5-(2-pyrazinyl)-1,2-dihydro-3-pyridinecarboxamide;
N-(5-((6,7-bis(methyloxy)-4-quinolinyl)oxy)-2-pyridinyl)-5-methyl-2-oxo-1-(phenylmethyl)-1,2-dihydro-3-pyridinecarboxamide;
N-(4-((6,7-bis(methyloxy)-4-quinolinyl)oxy)-3-fluorophenyl)-5-bromo-1-(3-methylphenyl)-2-oxo-1,2-dihydro-3-pyridinecarboxamide;
N-(4-((6,7-bis(methyloxy)-4-quinolinyl)oxy)-3-fluorophenyl)-5-(1-methyl-1H-pyrazol-4-yl)-2-oxo-1-phenyl-1,2-dihydro-3-pyridinecarboxamide;
N-(3-fluoro-4-((6-(methyloxy)-7-((3-(4-morpholinyl)propyl)oxy)-4-quinolinyl)oxy)phenyl)-2-oxo-5-phenyl-1-(phenylmethyl)-1,2-dihydro-3-pyridinecarboxamide;
1,1-dimethylethyl 5-(((5-((6,7-bis(methyloxy)-4-quinolinyl)oxy)-2-pyridinyl)amino)carbonyl)-6-oxo-1-(phenylmethyl)-1,3′,6,6′-tetrahydro-3,4′-bipyridine-1′(2′H)-carboxylate;
N-(4-((6,7-bis(methyloxy)-4-quinolinyl)oxy)-3-fluorophenyl)-2-oxo-1-(phenylmethyl)-5-(2-pyrimidinyl)-1,2-dihydro-3-pyridinecarboxamide;
N-(4-((6,7-bis(methyloxy)-4-quinolinyl)oxy)-3-fluorophenyl)-2-oxo-1-phenyl-5-(1H-pyrazol-4-yl)-1,2-dihydro-3-pyridinecarboxamide;
1-benzyl-5-bromo-N-(2-chloro-4-(6,7-dimethoxyquinolin-4-yloxy)phenyl)-2-oxo-1,2-dihydropyridine-3-carboxamide;
N-(5-(7-methoxyquinolin-4-yloxy)pyridin-2-yl)-2-oxo-1-phenyl-5-(pyridin-3-yl)-1,2-dihydropyridine-3-carboxamide;
N-(5-(7-methoxyquinolin-4-yloxy)pyridin-2-yl)-2-oxo-1-phenyl-5-(pyrazin-2-yl)-1,2-dihydropyridine-3-carboxamide;
N-(5-(6,7-dimethoxyquinolin-4-yloxy)pyridin-2-yl)-2-oxo-1-phenyl-5-(pyridin-3-yl)-1,2-dihydropyridine-3-carboxamide;
N-(5-(6,7-dimethoxyquinolin-4-yloxy)pyridin-2-yl)-2-oxo-1-phenyl-5-(pyrazin-2-yl)-1,2-dihydropyridine-3-carboxamide,
N-(5-(6,7-dimethoxyquinolin-4-yloxy)pyridin-2-yl)-2-oxo-1-phenyl-5-(thiophen-2-yl)-1,2-dihydropyridine-3-carboxamide;
5-benzyl-N-(5-(6,7-dimethoxyquinolin-4-yloxy)pyridin-2-yl)-2-oxo-1-phenyl-1,2-dihydropyridine-3-carboxamide;
tert-butyl 4-(5-((5-(6,7-dimethoxyquinolin-4-yloxy)pyridin-2-yl)carbamoyl)-6-oxo-1-phenyl-1,6-dihydropyridin-3-yl)-5,6-dihydropyridine-1(2H)-carboxylate;
5-bromo-N-(2-chloro-4-(6,7-dimethoxyquinolin-4-yloxy)phenyl)-2-oxo-1-phenyl-1,2-dihydropyridine-3-carboxamide;
N-(5-(6,7-dimethoxyquinolin-4-yloxy)pyridin-2-yl)-4-(2-methoxyethylamino)-2-oxo-1-phenyl-1,2-dihydropyridine-3-carboxamide;
N-(5-(6,7-dimethoxyquinolin-4-yloxy)pyridin-2-yl)-2-oxo-1-phenyl-4-(tetrahydro-2H-pyran-4-ylamino)-1,2-dihydropyridine-3-carboxamide;
N-(5-(6,7-dimethoxyquinolin-4-yloxy)pyridin-2-yl)-2-oxo-1-phenyl-4-(phenylamino)-1,2-dihydropyridine-3-carboxamide;
N-(5-(6,7-dimethoxyquinolin-4-yloxy)pyridin-2-yl)-4-(4-methylpiperazin-1-yl)-2-oxo-1-phenyl-1,2-dihydropyridine-3-carboxamide;
N-(5-(6,7-dimethoxyquinolin-4-yloxy)pyridin-2-yl)-4-(methylamino)-2-oxo-1-phenyl-1,2-dihydropyridine-3-carboxamide;
N-(5-(6,7-dimethoxyquinolin-4-yloxy)pyridin-2-yl)-4-(dimethylamino)-2-oxo-1-phenyl-1,2-dihydropyridine-3-carboxamide;
4-(2-methoxyethylamino)-N-(5-(7-methoxyquinolin-4-yloxy)pyridin-2-yl)-2-oxo-1-phenyl-1,2-dihydropyridine-3-carboxamide;
N-(3-fluoro-4-(7-methoxyquinolin-4-yloxy)phenyl)-4-(2-methoxyethylamino)-2-oxo-1-phenyl-1,2-dihydropyridine-3-carboxamide;
N-(4-((6,7-bis(methyloxy)-4-quinolinyl)oxy)-3-fluorophenyl)-1-cyclopentyl-6-oxo-5-(2-oxo-1-pyrrolidinyl)-1,6-dihydro-3-pyridinecarboxamide;
1-benzyl-N-(5-(6,7-dimethoxyquinolin-4-yloxy)pyridin-2-yl)-4-(2-methoxyethylamino)-2-oxo-1,2-dihydropyridine-3-carboxamide;
1-benzyl-N-(5-(6,7-dimethoxyquinolin-4-yloxy)pyridin-2-yl)-4-(dimethylamino)-2-oxo-1,2-dihydropyridine-3-carboxamide;
1-benzyl-N-(5-(6,7-dimethoxyquinolin-4-yloxy)pyridin-2-yl)-4-(methylamino)-2-oxo-1,2-dihydropyridine-3-carboxamide;
1-benzyl-N-(5-(6,7-dimethoxyquinolin-4-yloxy)pyridin-2-yl)-2-oxo-4-(phenylamino)-1,2-dihydropyridine-3-carboxamide;
1-benzyl-N-(5-(6,7-dimethoxyquinolin-4-yloxy)pyridin-2-yl)-2-oxo-4-(pyridin-4-ylamino)-1,2-dihydropyridine-3-carboxamide;
1-benzyl-N-(5-(6,7-dimethoxyquinolin-4-yloxy)pyridin-2-yl)-4-(4-methylpiperazin-1-yl)-2-oxo-1,2-dihydropyridine-3-carboxamide,
1-benzyl-N-(5-(6,7-dimethoxyquinolin-4-yloxy)pyridin-2-yl)-2-oxo-4-(tetrahydro-2H-pyran-4-ylamino)-1,2-dihydropyridine-3-carboxamide;
1-benzyl-N-(5-(6,7-dimethoxyquinolin-4-yloxy)pyridin-2-yl)-2-oxo-4-(4-(trifluoromethyl)phenylamino)-1,2-dihydropyridine-3-carboxamide;
1-cyclopentyl-N-(4-(6,7-dimethoxyquinolin-4-yloxy)-3-fluorophenyl)-6-oxo-5-(2-oxopyrrolidin-1-yl)-1,6-dihydropyridine-3-carboxamide;
N-(3-fluoro-4-(2-(pyrrolidine-1-carboxamido)pyridin-4-yloxy)phenyl)-3-oxo-2-phenyl-2,3-dihydropyridazine-4-carboxamide;
6-((diethylamino)methyl)-N-(4-(6,7-dimethoxyquinolin-4-yloxy)-3-fluorophenyl)-3-oxo-2-phenyl-2,3-dihydropyridazine-4-carboxamide;
6-((dimethylamino)methyl)-N-(3-fluoro-4-(7-methoxyquinolin-4-yloxy)phenyl)-3-oxo-2-phenyl-2,3-dihydropyridazine-4-carboxamide;
N-(3-fluoro-4-(7-methoxyquinolin-4-yloxy)phenyl)-6-methyl-3-oxo-2-phenyl-2,3-dihydropyridazine-4-carboxamide;
N-(5-(6,7-dimethoxyquinolin-4-yloxy)pyridin-2-yl)-6-methyl-3-oxo-2-phenyl-2,3-dihydropyridazine-4-carboxamide;
2-benzyl-N-(5-(6,7-dimethoxyquinolin-4-yloxy)pyridin-2-yl)-6-methyl-3-oxo-2,3-dihydropyridazine-4-carboxamide;
N-(3-fluoro-4-(7-methoxyquinolin-4-yloxy)phenyl)-3-oxo-2-phenyl-2,3-dihydropyridazine-4-carboxamide;
N-(2-chloro-4-(6,7-dimethoxyquinolin-4-yloxy)phenyl)-6-methyl-3-oxo-2-phenyl-2,3-dihydropyridazine-4-carboxamide;
(R)—N-(4-(6,7-dimethoxyquinolin-4-yloxy)-3-fluorophenyl)-6-((3-(dimethylamino)pyrrolidin-1-yl)methyl)-3-oxo-2-phenyl-2,3-dihydropyridazine-4-carboxamide;
3-benzyl-N-(4-(6,7-dimethoxyquinolin-4-yloxy)-3-fluorophenyl)-2-oxoimidazolidine-1-carboxamide;
N-(4-(6,7-dimethoxyquinolin-4-yloxy)-3-fluorophenyl)-5-((dimethylamino)methyl)-2-oxo-3-phenyl-tetrahydropyrimidine-1(2H)-carboxamide;
N-(3-Fluoro-4-(7-methoxyquinolin-4-yloxy)phenyl)-3-oxo-4-phenylmorpholine-2-carboxamide;
N-(5-(7-methoxyquinolin-4-yloxy)pyridin-2-yl)-1-methyl-3-oxo-2-phenyl-2,3-dihydro-1H-pyrazole-4-carboxamide; and
N-(3-Fluoro-4-(7-methoxyquinolin-4-yloxy)phenyl)-3-oxo-4-phenylmorpholine-2-carboxamide.

13. The method of claim 2, wherein at least one of the at least one HGF-Met inhibitor is selected from ARQ197, MK2461, PF2341066, XL880, and XL184.

14. The method of claim 2, wherein at least one of the at least one EGFR inhibitor is a specific binding agent to EGFR.

15. The method of claim 14, wherein the specific binding agent to EGFR is an antibody.

16. The method of claim 15, wherein the antibody is fully human.

17. The method of claim 15, wherein the antibody is selected from cetuximab and panitumumab.

18. The method of claim 17, wherein the antibody is cetuximab.

19. The method of claim 17, wherein the antibody is panitumumab.

20. The method of claim 15, wherein the antibody is administered in a dose of about 2 mg/kg to about 3 mg/kg per week, about 5 mg/kg to about 7 mg/kg every two weeks, or about 8 mg/kg to about 10 mg/kg every three weeks.

21. The method of claim 2, wherein at least one of the at least one HGF-Met inhibitor is a specific binding agent to HGF, and at least one of the at least one EGFR inhibitor is a specific binding agent to EGFR.

22. The method of claim 21, wherein the specific binding agent to HGF is an antibody to HGF, and the specific binding agent to EGFR is an antibody to EGFR.

23. The method of claim 22, wherein the antibody to HGF is 2.12.1.

24. The method of claim 22 or 23, wherein the antibody is EGFR is panitumumab.

25. The method of claim 2, wherein the cancer is a solid tumor.

26. The method of claim 2, wherein the cancer is selected from breast cancer, colorectal cancer, gastric carcinoma, glioblastoma, glioma cancer, head and neck cancer, hereditary and sporadic papillary renal carcinoma, leukemia, lymphoma, Li-Fraumeni syndrome, malignant pleural mesothelioma, medulloblastoma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

27. The method of claim 26, wherein the cancer is selected from breast cancer, colorectal cancer, gastric cancer, glioblastoma, head and neck cancer, non-small cell lung cancer, ovarian cancer, prostate cancer, and renal cell carcinoma.

28. The method of claim 27, wherein the cancer is glioblastoma.

29. A method of treating a resistant cancer in a patient comprising administering: (i) at least one HGF-Met inhibitor and at least one EGFR inhibitor; and (ii) at least one chemotherapy treatment.

30. The method of claim 29, wherein the at least one HGF-Met inhibitor and at least one EGFR inhibitor is administered prior to the administration of the chemotherapy treatment.

31. The method of claim 29, wherein the at least one HGF-Met inhibitor and at least one EGFR inhibitor is administered concurrent with the administration of the chemotherapy treatment.

32. The method of claim 29, wherein the at least one HGF-Met inhibitor and at least one EGFR inhibitor is administered subsequent to the administration of the chemotherapy treatment.

33. A method of treating a resistant cancer in a patient comprising administering: (i) at least one HGF-Met inhibitor and at least one EGFR inhibitor; and (ii) at least one radiation therapy.

34. The method of claim 33, wherein the at least one HGF-Met inhibitor and at least one EGFR inhibitor is administered prior to the administration of the radiation therapy.

35. The method of claim 33, wherein the at least one HGF-Met inhibitor and at least one EGFR inhibitor is administered concurrent with the administration of the radiation therapy.

36. The method of claim 33, wherein the at least one HGF-Met inhibitor and at least one EGFR inhibitor is administered subsequent to the administration of the radiation therapy.

37. A kit comprising at least one HGF-Met inhibitor and at least one EGFR inhibitor, wherein the at least one HGF-Met inhibitor and at least one EGFR inhibitor are in one or more containers.
